(19) United States
(12) Patent Application Publication
(10) Pub. No.: US 2018/0110704 A1
(43) Pub. Date: Apr. 26, 2018

(54) AEROSOL HAIR CARE COMPOSITIONS COMPRISING HFO FOAMING AGENT AND WATER MISCIBLE SOLVENTS

(71) Applicant: The Procter & Gamble Company, Cincinnati, OH (US)

(72) Inventors: Jean Jianqun Zhao, Cincinnati, OH (US); Ioannis Constantine Constantinides, Wyoming, OH (US); Karl Shiqing Wei, Mason, OH (US); Peter Herbert Koenig, Montgomery, OH (US); Howard David Hutton, III, Oregonia, OH (US); Robert Wayne Glenn, JR., Liberty Township, OH (US); Xiaoru Jenny Wang, Mason, OH (US); Douglas Charles Cook, South Lebanon, OH (US); Rootvij Dinesh Patel, Cincinnati, OH (US); Sarah Elizabeth Mullen, Cincinnati, OH (US); Jazmin Veronica Torres Rivera, Liberty Township, OH (US); Brian Xiaoqing Song, Mason, OH (US); Edward Dewey Smith, III, Mason, OH (US)

(21) Appl. No.: 15/789,172

(22) Filed: Oct. 20, 2017

Related U.S. Application Data
(60) Provisional application No. 62/411,060, filed on Oct. 21, 2016.

(51) Int. Cl. A61K 8/31; A61K 8/04; A61K 8/41; A61K 8/49; A61K 8/368; A61Q 5/02; A61Q 5/00

(52) U.S. Cl. CPC A61K 8/315; A61K 8/046; A61K 8/416; A61K 8/4926; A61K 8/494; A61K 2800/596; A61Q 5/02; A61Q 5/006; A61K 2800/87; A61K 2800/5424; A61K 2800/5426; A61K 8/368

(57) ABSTRACT

The invention relates to a hair care composition comprising: from about 5 weight % to about 45 weight % total surfactant; from about 5 weight % to about 35 weight % anionic surfactant; from about 1 to about 20% by weight of a water miscible solvent; greater than about 3 weight % HFO; wherein the ratio of HFO to water miscible solvent is lower than about 3, and wherein the foam density of the foam after dispensing is from about 0.03 to about 0.35 g/mL. The invention also relates to dispensing the composition from an aerosol container, wherein the foam density of the foam after dispensing is from about 0.03 to about 0.35 g/mL.

### FIELD OF THE INVENTION

The invention relates to an aerosol shampoo composition that comprises trans-1,3,3,3-tetrafluoropropene (“HFO”) and a water-miscible solvent.

### BACKGROUND OF THE INVENTION

Described herein are aerosol foams for the personal cleansing category represent an attractive form to the consumers. A shampoo product delivered via foam is readily spread on hair and enables hair cleansing without leaving significant residue on hair because the structuring effect of foam enables the use of compositions without polymeric or waxy structurants, resulting in cleaner and glossier hair. The development of plastic aerosol packages provides opportunities for developing attractive products in clear containers where the content of the package is visible to the consumer. Thus, clear products, which may signal to the consumer composition purity, are especially attractive for transparent packages and there is a need for developing transparent or translucent compositions.

Typical propellants or foaming agents for aerosol products are flammable volatile materials, such as hydrocarbons with low carbon chains. However, the content of such propellants or foaming agents in compositions delivered via plastic aerosols is limited because of their flammability. In addition, compositions containing hydrophobic foaming agents are often phase unstable, separating into two phases. Thus, shaking of the containers before dispensing is often needed. If the used propellant or foaming agent is biodegradable, the product becomes more sustainable. Thus, a need exists for cleansing aerosol compositions having non-flammable, biodegradable propellants or foaming agents that provide stable surfactant compositions.

Additionally, a shampoo product delivered via foam is an attractive consumer choice. However, because of the low density of the foam, high concentration of surfactant may be needed to deliver sufficient amount of detersive surfactant for each use. High surfactant liquid cleansing compositions often exhibit high viscosity, which makes it difficult to deliver with a typical aerosol foam dispenser. High surfactant liquid cleansing compositions are occasionally unstable and phase separate. Based on the foregoing, there is a need for a low viscosity, stable concentrated liquid cleansing composition for delivery as foam.

Trans-1,3,3,3-tetrafluoropropene (“HFO”) propellant or foaming agent is non-flammable and biodegradable and is compatible with many plastic containers. However, as with many compositions containing hydrophobic foaming agents, cleansing compositions containing HFO are occasionally unstable, because the hydrophobic material separates into a second phase. Thus, there is a need for the development of stable, single phase compositions using HFO propellant or foaming agent.

It has been surprisingly found that clear, stable, low viscosity compositions (even having high surfactant content) can be prepared by using the combination of HFO and a water-miscible solvent, such as dipropylene glycol, glycerin or propylene glycol, if the HFO:Solvent ratio is below 3. Such compositions can be delivered as foams using plastic (or other) aerosol packages.

### SUMMARY OF THE INVENTION

The invention relates to a hair care composition comprising: from about 5 weight % to about 45 weight % total surfactant; from about 5 weight % to about 35 weight % anionic surfactant; from about 1 to about 20% by weight of a water miscible solvent; greater than about 3% HFO; wherein the ratio of HFO to water miscible solvent is lower than about 3, and wherein the foam density after dispensing is between about 0.03 to about 0.35 g/mL. The hair care composition can be a shampoo, and the shampoo can be concentrated.

### DETAILED DESCRIPTION OF THE INVENTION

As used herein, the articles including “a” and “an” when used in a claim, are understood to mean one or more of what is claimed or described.

As used herein, “comprising” means that other steps and other ingredients which do not affect the end result can be added. This term encompasses the terms “consisting of” and “consisting essentially of”.

As used herein, “mixtures” is meant to include a simple combination of materials and any compounds that may result from their combination.

As used herein, “molecular weight” or “M.Wt.” refers to the weight average molecular weight unless otherwise stated. Molecular weight is measured using industry standard method, gel permeation chromatography (“GPC”).

As used herein, “personal care compositions” includes products such as shampoos, shower gels, liquid hand cleansers, hair colorants, facial cleansers, laundry detergent, dish detergent, and other surfactant-based liquid compositions

As used herein, the terms “include,” “includes,” and “including,” are meant to be non-limiting and are understood to mean “comprise,” “comprises,” and “comprising,” respectively.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Unless otherwise noted, all component or composition levels are in reference to the active portion of that component or composition, and are exclusive of impurities, for example, residual solvents or by-products, which may be present in commercially available sources of such components or compositions.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The hair care compositions described herein are cleaning aerosol compositions that comprise a non-flammable, inert, biodegradable propellant or foaming agent which provides a stable surfactant containing composition. The composition may be clear. The hair care composition can be a shampoo, and the shampoo can be concentrated. The hair care composition comprises from about 5 weight % to about 45 weight % total surfactant; from about 5 weight % to about 35 weight % anionic surfactant; from about 1 to about 20% by weight of a water miscible solvent; greater than about 3% HFO; wherein the ratio of HFO to water miscible solvent is lower than about 3, and wherein the foam density of the foam dispensed by the aerosol package is between about 0.03 to about 0.35 g/mL.

The ratio of HFO to water miscible solvent can be from about 0.1 to about 3, alternatively from about 0.1 to about 2.8.

A. Surfactants

The hair care composition can comprise a total surfactant level of from about 5% to about 45% by weight, from about 10% to about 45% by weight, from about 15% to about 45% by weight, from about 20% to about 45% by weigh, from about 20% to about 40% by weight. The total surfactants can include, but are not limited to anionic surfactants, amphoteric/zwitterionic surfactants, nonionic surfactants and combinations thereof. The hair care composition can comprise from about 5 weight % to about 35 weight % anionic surfactant.

Suitable anionic detersive surfactant components for use in the composition herein include those which are known for use in hair care or other personal care shampoo compositions. The anionic detersive surfactant may be a combination of sodium lauryl sulfate and sodium laureth-n sulfate. Alternatively, the anionic detersive surfactant can be sodium laureth sulfate with an average of one mole ethoxylate. The concentration of the anionic surfactant component in the composition should be sufficient to provide the desired cleaning and lather performance.

Anionic surfactants suitable for use herein include alkyl sulfates and alkyl ether sulfates of the formula ROSO₃M and RO(C₂H₄O)ₓSO₃M, wherein R is alkyl or alkenyl of from about 8 to about 18 carbon atoms, x is 1 to 10, and M is a water-soluble cation such as ammonium, sodium, potassium, and triethanolamine cation or salts of the divalent magnesium ion with two anionic surfactant anions. The alkyl ether sulfates may be made as condensation products of ethylene oxide and monohydric alcohols having from about 8 to about 24 carbon atoms. The alcohols can be derived from fats such as coconut oil, palm oil, palm kernel oil, or tallow, or can be synthetic.

TABLE 1
Examples of Typical Alkyl Sulfates and Alky Ether Sulfates

| Surfactant | Supplier | SLS | SLE1S | SLE2S | SLE3S | SLE > 3S |
|---|---|---|---|---|---|---|
| Sodium | Stepan | 100 | 0 | 0 | 0 | 0 |
| Lauryl | STEOL | | | | | |
| Sulfate | SLS | | | | | |
| Sodium | Stepan | 45.5 | 26.3 | 11.8 | 0.07 | 16.33 |
| Laureth-1 | STEOL | | | | | |
| Sulfate | SLES-1 | | | | | |
| Sodium | Stepan | 18 | 16.7 | 12.6 | 12.4 | 40.30 |
| Laureth-3 | STEOL | | | | | |
| Sulfate | SLES-3 | | | | | |

The composition of the present invention can also include anionic surfactants selected from the group consisting of:

a) R₁O(CH₂CHR₃O)_{y}S0₃M;

b) CH₃ (CH₂) CHR₂CH₂O(CH₂CHR₃O)_{z}S0₃M; and

c) mixtures thereof,

where R₁ represents CH₃ (CH₂)₁₀, R₂ represents H or a hydrocarbon radical comprising 1 to 4 carbon atoms such that the sum of the carbon atoms in z and R₂ is 8, R₃ is H or CH₃, y is to 7, the average value of y is about 1 when y is not zero (0), and M is a monovalent or divalent, positively-charged cation.

The composition can also include anionic alkyl sulfates and alkyl ether sulfate surfactants having branched alkyl chains which are synthesized from C8 to C18 2-alkylbranched alcohols which may be selected from the group consisting of: Guerbet alcohols, aldol alcohols, oxo alcohols and mixtures thereof. Nonlimiting examples of the 2-alkyl branched alcohols include the Guerbet alcohols such as 2-methyl-1-undecanol, 2-ethyl-1-decanol, 2-methyl-1-dodecanol, 2-butyl 1-octanol, 2-butyl-1-nonanol, 2-ethyl-1-undecanol, 2-propyl-1-nonanol, 2-pentyl-1-octanol, 2-pentyl-1-heptanol, and those sold under the tradename ISOFOL® (Sasol), and oxo alcohols, e.g., those sold under the tradenames LIAL® (Sasol), ISALCHEM® (Sasol), NEODOL® (Shell), Other suitable anionic surfactants include water-soluble salts of the organic, sulfonic acids of the general formula [R¹—SO₃M]. R¹ being a straight chain aliphatic hydrocarbon radical having from 13 to 17 carbon atoms, alternatively from 13 to 15 carbon atoms. M is a water soluble cation such as ammonium, sodium, potassium, and triethanolamine cation or salts of the divalent magnesium ion with two anionic surfactant anions. These materials are produced by the reaction of SO₂ and O₂ with suitable chain length normal paraffins (C₁₄-C₁₇) and are sold commercially as sodium paraffin sulfonates.

Some non-limiting examples of surfactants are:

Alkyl Sulfates

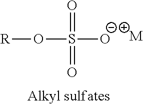

where R is C₈-C₂₄ alkyl (linear or branched, saturated or unsaturated) or mixtures thereof and M⁺ is monovalent cation. Examples include Sodium lauryl sulfate (where R is C₁₂ alkyl and M⁺ is Na⁺), ammonium lauryl sulfate (where R is C₁₂ alkyl and M⁺ is NH₃⁺), and sodium coco-sulfate (where R is coconut alkyl and M⁺ is Na⁺);

Alkyl Ether Sulfates

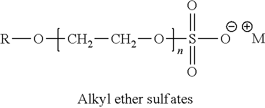

where R is C₈-C₂₄ alkyl (linear or branched, saturated or unsaturated) or mixtures thereof, n=1-12, and M⁺ is monovalent cation. Examples include sodium laureth sulfate (where R is C₁₂ alkyl and M⁺ is Na⁺, n=1-3), ammonium laureth sulfate (where R is C₁₂ alkyl, M⁺ is NH₃⁺, n=1-3), and Sodium trideceth sulfate (where R is C₁₃ alkyl, M⁺ is Na⁺, and n=1-4);

Some non-limiting examples of sulfonate surfactants are:

Alkyl glyceryl ether sulfonates:

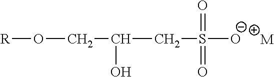

where R═C₈-C₂₄ alkyl (linear or branched, saturated or unsaturated) or mixtures thereof and M⁺=monovalent cation, such as Sodium Cocoglyceryl Ether Sulfonate (R=coco alkyl, M⁺=Na⁺);

Alpha olefin sulfonates prepared by sulfonation of long chain alpha olefins. Alpha olefin sulfonates consist of mixtures of alkene sulfonates,

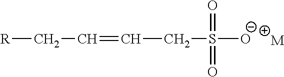

where R═C₈-C₁₈ alkyl or mixtures thereof and M⁺=monovalent cation;
Hydroxyalkyl sulfonates,

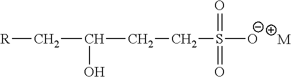

where R═C₄-C₁₈ alkyl or mixtures thereof and M⁺=monovalent cation. Examples include Sodium C12-14 Olefin Sulfonate (R═C₈-C₁₀ alkyl, M⁺=Na⁺) and Sodium C 14-16 Olefin Sulfonate (R═C₁₀-C₁₂ alkyl, M⁺=Na⁺).

Examples of additional anionic surfactants suitable for use herein include, but are not limited to, ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, sodium trideceth sulfate, sodium tridecyl sulfate, sodium methyl lauroyl taurate, sodium methyl cocoyl taurate, sodium lauroyl isethionate, sodium cocoyl isethionate, sodium laurethsulfosuccinate, sodium laurylsulfosuccinate, sodium tridecyl benzene sulfonate, sodium dodecyl benzene sulfonate, and mixtures thereof.

Additional anionic surfactants suitable for use herein include, but not limited to, acyl isethionate, acyl methyl isethionate, acyl glutamate, acyl glycinate, acyl sarcosinate, acyl alaninate, acyl taurate, sulfosuccinate, alkyl benzene sulfonate, alkyl ether carboxylate, alkylamphoacetate, alpha olefin sulfonate, and mixtures thereof. Examples of such suitable anionic surfactants include, but not limited to, sodium cocoyl isethionate, sodium lauroyl isethionate, sodium lauroyl methyl isethionate, sodium cocoyl glutamate, disodium cocoyl glutamate, sodium lauroyl glutamate, disodium lauroyl glutamate, sodium cocoyl alaninate, sodium lauroyl alaninate, sodium lauroyl glycinate, sodium cocoyl glycinate, sodium laureth sulfosuccinate, disodium laureth sulfosuccinate, sodium lauryl sulfosuccinate, disodium lauryl sulfosuccinate, sodium lauryl glucose carboxylate, sodium cocoyl glucose carboxylate, sodium cocoyl amphoacetate, sodium lauroyl amphoacetate, sodium methyl cocoyl taurate, and mixtures thereof.

The hair care composition comprises from about 0% to about 25%, from about 1% to about 23%, from about 2% to about 20%, from about 1% to about 20%, from about 1% to about 15%, from about 1% to about 10% by weight of one or more co-surfactants selected from the group consisting of amphoteric surfactant, zwitterionic surfactant, non-ionic surfactant and mixtures thereof.

Zwitterionic surfactants suitable for use herein include, but are not limited to derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one substituent contains an anionic group, e.g., carboxy, sulfonate, sulfate, phosphate, or phosphonate. Other zwitterionic surfactants suitable for use herein include betaines, including high alkyl betaines such as coco dimethyl carboxymethyl betaine, cocoamidopropyl betaine, cocobetaine, lauryl amidopropyl betaine, oleyl betaine, lauryl dimaethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, and mixtures thereof. The sulfobetaines may include coco dimethyl sulfopropyl betaine, stearyl dimethyl sulfopropyl betaine, lauryl dimethyl sulfoethyl betaine, lauryl bis-(2-hydroxyethyl) sulfopropyl betaine and mixtures thereof.

The suitable co-surfactants for use herein include zwitterionic molecules which possess a hydroxyl group along with positive and negative charges within the same molecule.

The suitable zwitterionic co-surfactants possess a hydroxyl group in their molecular structure are:

Alkyl Hydroxysultains

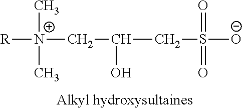

where R is alkyl group with C₈ to C₂₄ carbon chain (saturated or unsaturated) or mixture thereof. Examples include lauryl hydroxysultaine (where R is lauryl; C₁₂H₂₅) and coco-hydroxysultaine (where R is coco alkyl).

Alkylamidoalkyl Hydroxysultaines:

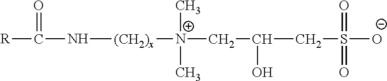

where RCO═C₆-C₂₄ acyl (saturated or unsaturated) or mixtures thereof. Examples include Cocamidopropyl Hydroxysultaine (RCO=coco acyl, x=3), Lauramidopropyl Hydroxysultaine (RCO=lauroyl, and x=3), Myristamidopropyl Hydroxysultaine (RCO=myristoyl, and x=3), and Oleamnidopropyl Hydroxysultaine (RCO=oleoyl, and x=3).

Alkyl Amphoacetates

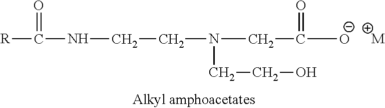

where R is alkyl group with C₆ to C₂, carbon chain (saturated or unsaturated) or mixtures thereof and M⁺ is monovalent cation. Examples include sodium lauroamphoacetate (where R is lauryl and M⁺ is Na⁺) and sodium cocoamphoacetate (where R is coco and M⁺ is Na⁺).

Alkyl Amphopropionates

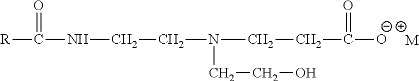

where RCO═C₆-C₂₄ acyl (saturated or unsaturated) or mixtures thereof and M⁺=monovalent cation. Examples include Sodium Lauroamphopropionate (RCO=lauroyl and M⁺=Na⁺) and Sodium Cocoamphopropionate (RCO=coco acyl and M⁺=Na⁺).

Alkyl Amphohydroxypropylsulfonates:

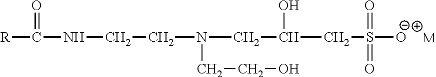

where RCO═C₆-C₂₄ acyl (saturated or unsaturated) or mixtures thereof and M⁺=monovalent cation, Examples include Sodium Lauroamphohydroxypropylsulfonate (RCO=lauroyl and M⁺=Na⁺) and Sodium Cocoamphohydroxypropylsulfonate (RCO=coco acyl and M⁺=Na⁺).

Alkyl Phosphobetaines:

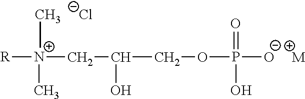

where R═C₆-C₂₄ alkyl (saturated or unsaturated) or mixtures thereof and M⁺=monovalent cation, such as Sodium Coco PG-Dimonium Chloride Phosphate, where R=coco alkyl and M⁺=Na⁺

Amphohydroxyalkylphosphates of the Formula:

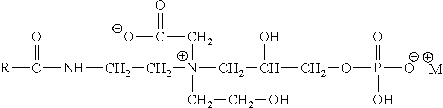

Suitable anionic surfactant can be surfactant with a tail having an alkyl chain with 8 carbon atoms or higher, include, but are not limited to the following surfactants: sodium trideceth sulfate, sodium tridecyl sulfate, sodium C8-13 alkyl sulfate, sodium C8-15 alkyl sulfate, sodium C8-18 alkyl sulfate, sodium C8-13 pareth sulfate, sodium C8-13 pareth-n sulfate, sodium C8-14 pareth-n sulfate, and combinations thereof. Other salts of all the aforementioned surfactants are useful, such as TEA, DEA, ammonia, potassium salts. Useful alkoxylates include the ethylene oxide, propylene oxide and EO/PO mixed alkoxylates. Phosphates, carboxylates and sulfonates prepared from branched alcohols are also useful anionic branched surfactants. Branched surfactants can be derived from synthetic alcohols such as the primary alcohols from the liquid hydrocarbons produced by Fischer-Tropsch condensed syngas, for example Safol™ 23 Alcohol available from Sasol North America, Houston, Tex.; from synthetic alcohols such as Neodol™ 23 Alcohol available from Shell Chemicals, USA; from synthetically made alcohols such as those described in U.S. Pat. No. 6,335,312 issued to Coffindaffer, et al on Jan. 1, 2002. Suitable examples of alcohols are Safol™ 23 and Neodol™ 23. Suitable examples of alkoxylated alcohols are Safol™ 23-3 and Neodol™ 23-3. Sulfates can be prepared by conventional processes to high purity from a sulfur based SO3 air stream process, chlorosulfonic acid process, sulfuric acid process, or Oleum process. Preparation via air stream in a falling film reactor is a preferred sulfation process. The anionic surfactant may also be STnS, wherein n can define average moles of ethoxylation. n can range from about 0 to about 3.5, from about 0.5 to about 3.5, from about 1.1 to about 3.5, from about 1.8 to about 3, or n can be about 2 or 3.

The hair care composition can comprise from about 0% to about 15% by weight, from about 0.5% to about 12% by weight, from about 1% to about 12% by weight, from about 1% to about 8% by weight, from about 0% to about 10% by weight, from about 0.5% to about 10% by weight from about 1% to about 10% by weight, from about 2% to about 5% by weight and from about 2% to about 12% by weight of an amphoteric surfactant. Various examples and descriptions of detersive surfactants are set forth in U.S. Pat. No. 8,440,605; U.S. Patent Application Publication No. 2009/155383; and U.S. Patent Application Publication No. 2009/0221463, which are incorporated herein by reference in their entirety.

Amphoteric detersive surfactants suitable for use in the hair care composition include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Exemplary amphoteric detersive surfactants for use in the present hair care composition include sodium cocoamphoacetate, sodium cocoamphodiacetate, sodium lauroamphoacetate, disodium lauroamphodiacetate, sodium cocaminopropionate, sodium cocaminodipropionate, sodium cocoamphohydroxypropylsulfonate, sodium cocoamphopropionate, sodium cornamphopropionate, sodium lauraminopropionate, sodium lauroarnphohydroxypropylsulfonate, sodium lauroamphopropionate, sodium cornamphopropionate, sodium lauriminodipropionate, ammonium cocaminoproppionate, ammonium cocaminodipropionate, ammonium cocoamphoacetate, ammonium cocoamphohydroxypropylsulfonate, ammonium cocoamphopropionate, ammonium cornamphopropionate, ammonium lauraminopropionate, ammonium lauroamphoacetate, ammonium lauroamphohydroxypropylsulfonate, ammonium lauroamphopropionate, ammonium cornamphopropionate, ammonium lauriminodipropionate, triethanonlamine cocaminopropionate, triethanonlamine cocaminodipropionate, triethanonlamine cocoamphoacetate, triethanonlamine cocooamphohydroxypropyl sulfonate, triethanonlamine cocoamphopropionate, triethanonlamine cornamphopropionate, triethanonlamine lauraminopropionate, triethanonlamine lauroamphoacetate, triethanonlamine lauroamphohydroxypropylsulfonate, triethanonlamine lauroarnphoproppionate, triethanonlamine cornamphopropionate, triethanonlamine lauriminodipropionate, cocoamphodipropionic acid, disodium caproamphodiacetate, disodium caproarmphoadipropionate, disodium capryloamphodiacetate, disodium capryloarnphodipriopionate, disodium cocoamphocarboxyethihydroxypropylsulfonate, disodium cocoamphodiacetate, disodium cocoamphodipropionate, disodium dicarboxyethylcocopropylenediamine, disodium laureth-5 carboxyarmphodiacetate, disodium lauriminodipropionate, disodium lauroamphodipropionate, disodium oleoamphodipropionate, disodium PPG-2-isodecethyl-7 carboxyamphodiacetate, lauraminopropionic acid, lauroamphodipropionic acid, lauryl aminopropylglycine, lauryl diethylenediaminoglycine, and mixtures thereof. Also suitable amphoteric surfactants include amidobetaines and amidosulfobetaines, wherein the RCONH(CH₂)₃ radical, wherein R is a C₁₁-C17 alkyl, is attached to the nitrogen atom of the betaine are also useful in this invention. The hair care composition can comprise from about 0% to about 15% by weight, from about 0.5% to about 12% by weight, from about 1% to about 12% by weight, from about 1% to about 8% by weight, and from about 2% to about 12% by weight from about 0% to about 10% by weight, from about 0.5% to about 10% by weight from about 1% to about 10% by weight and from about 2% to about 5% by weight of an non-ionic surfactant.

Suitable non-ionic surfactants can be selected from the group consisting of: Cocamide, Cocamide Methyl MEA, Cocamide DEA, Cocamide MEA, Cocamide MIPA, Lauramide DEA, Lauramide MEA, Lauramide MIPA, Myristamide DEA, Myristamide MEA, PEG-20 Cocamide MEA, PEG-2 Cocamide, PEG-3 Cocamide, PEG-4 Cocamide, PEG-5 Cocamide, PEG-6 Cocamide, PEG-7 Cocamide, PEG-3 Lauramide, PEG-5 Lauramide, PEG-3 Oleamide, PPG-2 Cocamide, PPG-2 Hydroxyethyl Cocamide, and mixtures thereof.

Suitable nonionic surfactants for use include those described in McCutcheon's Detergents and Emulsifiers, North American edition (1986), Allured Publishing Corp., and McCutcheon's Functional Materials, North American edition (1992). Suitable nonionic surfactants for use in the hair care compositions include, but are not limited to, polyoxyethylenated alkyl phenols, polyoxyethylenated alcohols, polyoxyethylenated polyoxypropylene glycols, glyceryl esters of alkanoic acids, polyglyceryl esters of alkanoic acids, propylene glycol esters of alkanoic acids, sorbitol esters of alkanoic acids, polyoxyethylenated sorbitor esters of alkanoic acids, polyoxyethylene glycol esters of alkanoic acids, polyoxyethylenated alkanoic acids, alkanolamides, N-alkylpyrrolidones, alkyl glycosides, alkyl polyglucosides, alkylamine oxides, and polyoxyethylenated silicones.

Representative polyoxyethylenated alcohols include alkyl chains ranging in the C9-C16 range and having from about 1 to about 110 alkoxy groups including, but not limited to, laureth-3, laureth-23, ceteth-10, steareth-10, steareth-100, beheneth-10, and commercially available from Shell Chemicals, Houston, Tex. under the trade names Neodol® 91, Neodol® 23, Neodol® 25, Neodol® 45, Neodol® 135, Neodo® l 67, Neodol® PC 100, Neodol® PC 200, Neodol® PC 600, and mixtures thereof.

Also available commercially are the polyoxyethylene fatty ethers available commercially under the Brij® trade name from Uniqema, Wilmington, Del., including, but not limited to, Brij® 30, Brij® 35, Brij® 52, Brij® 56, Brij® 58, Brij® 72, Brij® 76, Brij® 78, Brij® 93, Brij® 97, Brij® 98, Brij® 721 and mixtures thereof.

Suitable alkyl glycosides and alkyl polyglucosides can be represented by the formula (S)n-O—R wherein S is a sugar moiety such as glucose, fructose, mannose, galactose, and the like; n is an integer of from about 1 to about 1000, and R is a C8-C30 alkyl group. Examples of long chain alcohols from which the alkyl group can be derived include decyl alcohol, lauryl alcohol, myristyl alcohol, cetyl alcohol, stearyl alcohol, oleyl alcohol, and the like. Examples of these surfactants include alkyl polyglucosides wherein S is a glucose moiety, R is a C8-20 alkyl group, and n is an integer of from about 1 to about 9. Commercially available examples of these surfactants include decyl polyglucoside and lauryl polyglucoside available under trade names APG® 325 CS, APG® 600 CS and APG® 625 CS) from Cognis, Ambler, Pa. Also useful herein are sucrose ester surfactants such as sucrose cocoate and sucrose laurate and alkyl polyglucosides available under trade names Triton™ BG-10 and Triton™ CG-110 from The Dow Chemical Company, Houston, Tex.

Other nonionic surfactants suitable for use are glyceryl esters and polyglyceryl esters, including but not limited to, glyceryl monoesters, glyceryl monoesters of C12-22 saturated, unsaturated and branched chain fatty acids such as glyceryl oleate, glyceryl monostearate, glyceryl monopalmitate, glyceryl monobehenate, and mixtures thereof, and polyglyceryl esters of C12-22 saturated, unsaturated and branched chain fatty acids, such as polyglyceryl-4 isostearate, polyglyceryl-3 oleate, polyglyceryl-2-sesquioleate, triglyceryl diisostearate, diglyceryl monooleate, tetraglyceryl monooleate, and mixtures thereof.

Also useful herein as nonionic surfactants are sorbitan esters. Sorbitan esters of C12-22 saturated, unsaturated, and branched chain fatty acids are useful herein. These sorbitan esters usually comprise mixtures of mono-, di-, tri-, etc. esters. Representative examples of suitable sorbitan esters include sorbitan monolaurate (SPAN® 20), sorbitan monopalmitate (SPAN® 40), sorbitan monostearate (SPAN® 60), sorbitan tristearate (SPAN® 65), sorbitan monooleate (SPAN® 80), sorbitan trioleate (SPAN® 85), and sorbitan isostearate.

Also suitable for use herein are alkoxylated derivatives of sorbitan esters including, but not limited to, polyoxyethylene (20) sorbitan monolaurate (Tween® 20), polyoxyethylene (20) sorbitan monopalmitate (Tween® 40), polyoxyethylene (20) sorbitan monostearate (Tween® 60), polyoxyethylene (20) sorbitan monooleate (Tween® 80), polyoxyethylene (4) sorbitan monolaurate (Tween® 21), polyoxyethylene (4) sorbitan monostearate (Tween® 61), polyoxyethylene (5) sorbitan monooleate (Tween® 81), and mixtures thereof, all available from Uniqema.

Also suitable for use herein are alkylphenol ethoxylates including, but not limited to, nonylphenol ethoxylates (Tergitol™ NP-4, NP-6, NP-7, NP-8, NP-9, NP-10, NP-11, NP-12, NP-13, NP-15, NP-30, NP-40, NP-50, NP-55, NP-70 available from The Dow Chemical Company, Houston, Tex.) and octylphenol ethoxylates (Triton™ X-15, X-35, X-45, X-114, X-100, X-102, X-165, X-305, X-405, X-705 available from The Dow Chemical Company, Houston, Tx).

Also suitable for use herein are alkanolamides including cocamide monoethanolamine (CMEA) and tertiary alkylamine oxides including lauramine oxide and cocamine oxide.

Nonionic surfactants useful herein have an HLB (hydrophile-lipophile balance) of at least 8, in one embodiment greater than 10, and in another embodiment greater than 12. The HLB represents the balance between the hydrophilic and lipophilic moieties in a surfactant molecule and is commonly used as a method of classification. The HLB values for commonly-used surfactants are readily available in the literature (e.g., HLB Index in McCutcheon's Emulsifiers and Detergents, MC Publishing Co., 2004).

Non limiting examples of other anionic, zwitterionic, amphoteric, and non-ionic additional surfactants suitable for use in the hair care composition are described in McCutcheon's, Emulsifiers and Detergents, 1989 Annual, published by M. C. Publishing Co., and U.S. Pat. Nos. 3,929,678, 2,658,072; 2,438,091; 2,528,378, which are incorporated herein by reference in their entirety.

B. Water Miscible Solvent

The hair care composition comprises water-miscible solvent or combination of water-miscible solvent. The content of the water-miscible solvent is from about 1 wt % to about 20 wt %, from about 2 wt % to about 20 wt %, from about 2 wt % to about 18 wt %, from about 2 wt % to about 15 wt %, from about 4 wt % to about 15 wt %, from about 4 wt % to about 10 wt %. Suitable water miscible solvents include, but are not limited to, dipropylene glycol, tripropylene glycol, diethylene glycol, ethylene glycol, propylene glycol, glycerin, 1,3-propane diol, 2,2-propanediol, 1,2-butanediol, 1,3-butanediol, 1,4-butanediol, 2,3-butanediol, 2-methyl-2,4-pentanediol, and mixtures thereof. The hair care composition may comprise two or more water miscible solvents, wherein at least one of the solvents is dipropylene glycol.

The hair care compositions may have a pH in the range from about 2 to about 10, at 25° C. Alternatively, the hair care composition has a pH in the range from about −4 to about 7, which may help to solubilize minerals and redox metals already deposited on the hair. Thus, the hair care composition can also be effective toward washing out the existing minerals and redox metals deposits, which can reduce cuticle distortion and thereby reduce cuticle chipping and damage.

C. Water Carrier

The compositions can include from about 45% to about 78% by weight, from about 50% to about 75%, from about 55% to about 70% water, from about 60% to about 68% by weight of water.

D. Cationic Polymers

The hair care composition also comprises a cationic polymer. These cationic polymers can include at least one of (a) a cationic guar polymer, (b) a cationic non-guar galactomannan polymer, (c) a cationic tapioca polymer, (d) a cationic copolymer of acrylamide monomers and cationic monomers, and/or (e) a synthetic, non-crosslinked, cationic polymer, which may or may not form lyotropic liquid crystals upon combination with the detersive surfactant (f) a cationic cellulose polymer. Additionally, the cationic polymer can be a mixture of cationic polymers.

The hair care composition may comprise a cationic guar polymer, which is a cationically substituted galactomannan (guar) gum derivatives. Guar gum for use in preparing these guar gum derivatives is typically obtained as a naturally occurring material from the seeds of the guar plant. The guar molecule itself is a straight chain mannan, which is branched at regular intervals with single membered galactose units on alternative mannose units. The mannose units are linked to each other by means of β(1-4) glycosidic linkages. The galactose branching arises by way of an α(1-6) linkage. Cationic derivatives of the guar gums are obtained by reaction between the hydroxyl groups of the polygalactomannan and reactive quaternary ammonium compounds. The degree of substitution of the cationic groups onto the guar structure should be sufficient to provide the requisite cationic charge density described above.

The cationic polymer, can include, but not limited, to a cationic guar polymer, has a molecular weight of less than 2.0 million g/mol, or from about 10 thousand to about 2 million g/mol, or from about 50 thousand to about 2 million g/mol, or from about 100 thousand to about 2 million g/mol, or from about 10 thousand to about 1 million g/mol, or from about 25 thousand to about 1 million g/mol, or from about 50 thousand to about 1 million g/mol, or from about 100 thousand to about 1 million g/mol. The cationic guar polymer can have a charge density of from about 0.2 to about 2.2 meq/g, or from about 0.3 to about 2.0 meq/g, or from about 0.4 to about 1.8 meq/g; or from about 0.5 meq/g to about 1.7 meq/g.

The cationic guar polymer can have a weight average molecular weight of less than about 1.0 million g/mol, and has a charge density of from about 0.1 meq/g to about 2.5 meq/g. In an embodiment, the cationic guar polymer has a weight average molecular weight of less than 950 thousand g/mol, or from about 10 thousand to about 900 thousand g/mol, or from about 25 thousand to about 900 thousand g/mol, or from about 50 thousand to about 900 thousand g/mol, or from about 100 thousand to about 900 thousand g/mol. from about 150 thousand to about 800 thousand g/mol. The cationic guar polymer can have a charge density of from about 0.2 to about 2.2 meq/g, or from about 0.3 to about 2.0 meq/g, or from about 0.4 to about 1.8 meq/g; or from about 0.5 meq/g to about 1.5 meq/g.

The hair care composition can comprise from about 0.05% to less than about 1%, from about 0.05% to about 0.9%, from about 0.1% to about 0.8%, or from about 0.2% to about 0.7% of cationic polymer (a), by total weight of the composition.

The cationic guar polymer may be formed from quaternary ammonium compounds. The quaternary ammonium compounds for forming the cationic guar polymer can conform to the general formula 1:

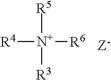

wherein where R³, R⁴ and R⁵ are methyl or ethyl groups; R⁶ is either an epoxyalkyl group of the general formula 2:

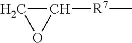

or R⁶ is a halohydrin group of the general formula 3:

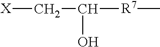

wherein R⁷ is a C₁ to C₃ alkylene; X is chlorine or bromine, and Z is an anion such as Cl—, Br—, I— or HSO₄—.

The cationic guar polymer can conform to the general formula 4:

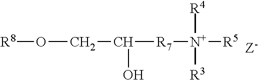

wherein R⁸ is guar gum; and wherein R⁴, R⁵, R⁶ and R⁷ are as defined above; and wherein Z is a halogen. The cationic guar polymer can conform to Formula 5:

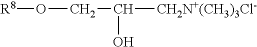

Suitable cationic guar polymers include cationic guar gum derivatives, such as guar hydroxypropyltrimonium chloride. The cationic guar polymer can be a guar hydroxypropyltrimonium chloride. Specific examples of guar hydroxypropyltrimonium chlorides include the Jaguar® series commercially available from Rhone-Poulenc Incorporated, for example Jaguar® C-500, commercially available from Rhodia. Jaguar® C-500 has a charge density of 0.8 meq/g and a molecular weight of 500,000 g/mol. Other suitable guar hydroxypropyltrimonium chloride are: guar hydroxypropyltrimonium chloride which has a charge density of about 1.1 meq/g and a molecular weight of about 500,000 g/mol is available from ASI, a charge density of about 1.5 meq/g and a molecular weight of about 500,000 g/mole is available from ASI. Other suitable guar hydroxypropyltrimonium chloride are: Hi-Care 1000, which has a charge density of about 0.7 meq/g and a Molecular weight of about 600,000 g/mole and is available from Rhodia; N-Hance 3269 and N-Hance 3270, which has a charge density of about 0.7 meq/g and a molecular weight of about 425,000 g/mol and is available from ASIAquaCat CG518 has a charge density of about 0.9 meq/g and a Molecular weight of about 50,000 g/mol and is available from ASI. BF-13, which is a borate (boron) free guar of charge density of about 1.1 meq/g and molecular weight of about 800,000 and BF-17, which is a borate (boron) free guar of charge density of about 1.7 meq/g and M. W.t of about 800,000 both available from ASI.

The hair care compositions may comprise a galactomannan polymer derivative having a mannose to galactose ratio of greater than 2:1 on a monomer to monomer basis, the galactomannan polymer derivative selected from the group consisting of a cationic galactomannan polymer derivative and an amphoteric galactomannan polymer derivative having a net positive charge. As used herein, the term “cationic galactomannan” refers to a galactomannan polymer to which a cationic group is added. The term “amphoteric galactomannan” refers to a galactomannan polymer to which a cationic group and an anionic group are added such that the polymer has a net positive charge.

Galactomannan polymers are present in the endosperm of seeds of the Leguminosae family. Galactomannan polymers are made up of a combination of mannose monomers and galactose monomers. The galactomannan molecule is a straight chain mannan branched at regular intervals with single membered galactose units on specific mannose units. The mannose units are linked to each other by means of β (1-4) glycosidic linkages. The galactose branching arises by way of an α (1-6) linkage. The ratio of mannose monomers to galactose monomers varies according to the species of the plant and also is affected by climate. Non Guar Galactomannan polymer derivatives suitable for use can have a ratio of mannose to galactose of greater than 2:1 on a monomer to monomer basis. Suitable ratios of mannose to galactose can be greater than about 3:1, and the ratio of mannose to galactose can be greater than about 4:1. Analysis of mannose to galactose ratios is well known in the art and is typically based on the measurement of the galactose content.

The gum for use in preparing the non-guar galactomannan polymer derivatives is typically obtained as naturally occurring material such as seeds or beans from plants. Examples of various non-guar galactomannan polymers include but are not limited to Tara gum (3 parts mannose/1 part galactose), Locust bean or Carob (4 parts mannose/1 part galactose), and Cassia gum (5 parts mannose/1 part galactose).

The non-guar galactomannan polymer derivatives can have a M. Wt. from about 1,000 to about 1,000,000, and/or form about 5,000 to about 900,000.

The hair care compositions of the can also include galactomannan polymer derivatives which have a cationic charge density from about 0.5 meq/g to about 7 meq/g. The galactomannan polymer derivatives can have a cationic charge density from about 1 meq/g to about 5 meq/g. The degree of substitution of the cationic groups onto the galactomannan structure should be sufficient to provide the requisite cationic charge density.

The galactomannan polymer derivative can be a cationic derivative of the non-guar galactomannan polymer, which is obtained by reaction between the hydroxyl groups of the polygalactomannan polymer and reactive quaternary ammonium compounds. Suitable quaternary ammonium compounds for use in forming the cationic galactomannan polymer derivatives include those conforming to the general formulas 1-5, as defined above.

Cationic non-guar galactomannan polymer derivatives formed from the reagents described above are represented by the general formula 6:

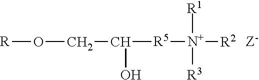

wherein R is the gum. The cationic galactomannan derivative can be a gum hydroxypropyltrimethylammonium chloride, which can be more specifically represented by the general formula 7:

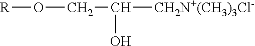

Alternatively the galactomannan polymer derivative can be an amphoteric galactomannan polymer derivative having a net positive charge, obtained when the cationic galactomannan polymer derivative further comprises an anionic group.

The cationic non-guar galactomannan can have a ratio of mannose to galactose is greater than about 4:1, a molecular weight of about 50,000 g/mol to about 1,000,000 g/mol, and/or from about 100,000 g/mol to about 900,000 g/mol and a cationic charge density from about 1 meq/g to about 5 meq/g, and/or from 2 meq/g to about 4 meq/g and can also be derived from a cassia plant.

The hair care compositions can comprise at least about 0.05% of a galactomannan polymer derivative by weight of the composition, alternatively from about 0.05% to about 2%, by weight of the composition, of a galactomannan polymer derivative.

The hair care compositions can comprise water-soluble cationically modified starch polymers. As used herein, the term “cationically modified starch” refers to a starch to which a cationic group is added prior to degradation of the starch to a smaller molecular weight, or wherein a cationic group is added after modification of the starch to achieve a desired molecular weight. The definition of the term “cationically modified starch” also includes amphoterically modified starch. The term “amphoterically modified starch” refers to a starch hydrolysate to which a cationic group and an anionic group are added.

The hair care compositions can comprise cationically modified starch polymers at a range of about 0.01% to about 10%, and/or from about 0.05% to about 5%, by weight of the composition.

The cationically modified starch polymers disclosed herein have a percent of bound nitrogen of from about 0.5% to about 4%.

The cationically modified starch polymers for use in the hair care compositions can have a molecular weight about 50,000 g/mol to about 1,000,000 g/mol and/or from about 100,000 g/mol to about 1,000,000 g/mol.

The hair care compositions can include cationically modified starch polymers which have a charge density of from about 0.2 meq/g to about 5 meq/g, and/or from about 0.2 meq/g to about 2 meq/g. The chemical modification to obtain such a charge density includes, but is not limited to, the addition of amino and/or ammonium groups into the starch molecules. Non-limiting examples of these ammonium groups may include substituents such as hydroxypropyl trimmonium chloride, trimethylhydroxypropyl ammonium chloride, dimethylstearylhydroxypropyl ammonium chloride, and dimethyldodecylhydroxypropyl ammonium chloride. See Solarek, D. B., Cationic Starches in Modified Starches: Properties and Uses, Wurzburg, O. B., Ed., CRC Press, Inc., Boca Raton, Fla. 1986, pp 113-125. The cationic groups may be added to the starch prior to degradation to a smaller molecular weight or the cationic groups may be added after such modification.

The cationically modified starch polymers generally have a degree of substitution of a cationic group from about 0.2 to about 2.5. As used herein, the “degree of substitution” of the cationically modified starch polymers is an average measure of the number of hydroxyl groups on each anhydroglucose unit which is derivatized by substituent groups. Since each anhydroglucose unit has three potential hydroxyl groups available for substitution, the maximum possible degree of substitution is 3. The degree of substitution is expressed as the number of moles of substituent groups per mole of anhydroglucose unit, on a molar average basis. The degree of substitution may be determined using proton nuclear magnetic resonance spectroscopy (“.sup.1H NMR”) methods well known in the art. Suitable .sup.1H NMR techniques include those described in “Observation on NMR Spectra of Starches in Dimethyl Sulfoxide, Iodine-Complexing, and Solvating in Water-Dimethyl Sulfoxide”, Qin-Ji Peng and Arthur S. Perlin, Carbohydrate Research, 160 (1987), 57-72; and “An Approach to the Structural Analysis of Oligosaccharides by NMR Spectroscopy”, J. Howard Bradbury and J. Grant Collins, Carbohydrate Research, 71, (1979), 15-25.

The source of starch before chemical modification can be chosen from a variety of sources such as tubers, legumes, cereal, and grains. Non-limiting examples of this source starch may include corn starch, wheat starch, rice starch, waxy corn starch, oat starch, cassava starch, waxy barley, waxy rice starch, glutenous rice starch, sweet rice starch, amioca, potato starch, tapioca starch, oat starch, sago starch, sweet rice, or mixtures thereof.

The cationically modified starch polymers can be selected from degraded cationic maize starch, cationic tapioca, cationic potato starch, and mixtures thereof. Alternatively, the cationically modified starch polymers are cationic corn starch and cationic tapioca.

The starch, prior to degradation or after modification to a smaller molecular weight, may comprise one or more additional modifications. For example, these modifications may include cross-linking, stabilization reactions, phosphorylations, and hydrolyzations. Stabilization reactions may include alkylation and esterification.

The cationically modified starch polymers may be incorporated into the composition in the form of hydrolyzed starch (e.g., acid, enzyme, or alkaline degradation), oxidized starch (e.g., peroxide, peracid, hypochlorite, alkaline, or any other oxidizing agent), physically/mechanically degraded starch (e.g., via the thermo-mechanical energy input of the processing equipment), or combinations thereof.

An optimal form of the starch is one which is readily soluble in water and forms a substantially clear (% Transmittance.gtoreq.80 at 600 nm) solution in water. The transparency of the composition is measured by Ultra-Violet/Visible (UV/VIS) spectrophotometry, which determines the absorption or transmission of UV/VIS light by a sample, using a Gretag Macbeth Colorimeter Color i 5 according to the related instructions. A light wavelength of 600 nm has been shown to be adequate for characterizing the degree of clarity of cosmetic compositions.

Suitable cationically modified starch for use in hair care compositions are available from known starch suppliers. Also suitable for use in hair care compositions are nonionic modified starch that can be further derivatized to a cationically modified starch as is known in the art. Other suitable modified starch starting materials may be quaternized, as is known in the art, to produce the cationically modified starch polymer suitable for use in hair care compositions.

Starch Degradation Procedure: a starch slurry can be prepared by mixing granular starch in water. The temperature is raised to about 35° C. An aqueous solution of potassium permanganate is then added at a concentration of about 50 ppm based on starch. The pH is raised to about 11.5 with sodium hydroxide and the slurry is stirred sufficiently to prevent settling of the starch. Then, about a 30% solution of hydrogen peroxide diluted in water is added to a level of about 1% of peroxide based on starch. The pH of about 11.5 is then restored by adding additional sodium hydroxide. The reaction is completed over about a 1 to about 20 hour period. The mixture is then neutralized with dilute hydrochloric acid. The degraded starch is recovered by filtration followed by washing and drying.

The hair care composition can comprise a cationic copolymer of an acrylamide monomer and a cationic monomer, wherein the copolymer has a charge density of from about 1.0 meq/g to about 3.0 meq/g. The cationic copolymer can be a synthetic cationic copolymer of acrylamide monomers and cationic monomers.

The cationic copolymer can comprise:
(i) an acrylamide monomer of the following Formula AM:

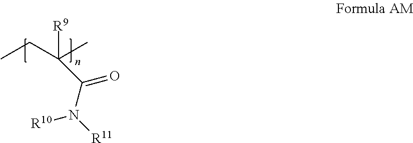

where R⁹ is H or C₁₋₄ alkyl; and R¹⁰ and R¹¹ are independently selected from the group consisting of H, C₁₋₄ alkyl, CH₂OCH₃, CH₂OCH₂CH(CH₃)₂, and phenyl, or together are C₃-6cycloalkyl; and
(ii) a cationic monomer conforming to Formula CM:

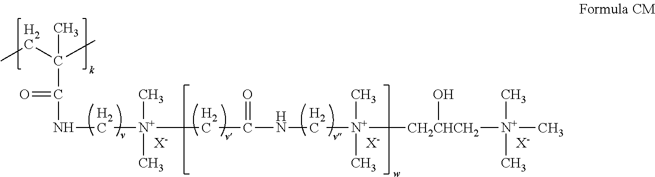

where k=1, each of v, v′, and v″ is independently an integer of from 1 to 6, w is zero or an integer of from 1 to 10, and X⁻ is an anion.

The cationic monomer can conform to Formula CM and where k=1, v=3 and w=0, z=1 and X⁻ is Cl⁻ to form the following structure:

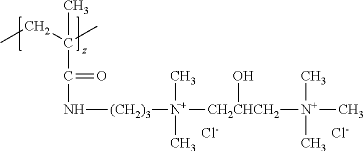

The above structure may be referred to as diquat. Alternatively, the cationic monomer can conform to Formula CM and wherein v and v″ are each 3, v′=1, w=1, y=1 and X⁻ is Cl⁻, such as:

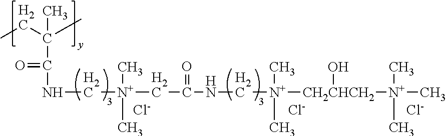

The above structure may be referred to as triquat.

Suitable acrylamide monomer include, but are not limited to, either acrylamide or methacrylamide.

The cationic copolymer can be of an acrylamide monomer and a cationic monomer, wherein the cationic monomer is selected from the group consisting of: dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide; ethylenimine, vinylamine, 2-vinylpyridine, 4-vinylpyridine; trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride, and mixtures thereof.

The cationic copolymer can comprise a cationic monomer selected from the group consisting of: cationic monomers include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, and mixtures thereof.

The cationic copolymer can be water-soluble. The cationic copolymer is formed from (1) copolymers of (meth)acrylamide and cationic monomers based on (meth)acrylamide, and/or hydrolysis-stable cationic monomers, (2) terpolymers of (meth)acrylamide, monomers based on cationic (meth)acrylic acid esters, and monomers based on (meth)acrylamide, and/or hydrolysis-stable cationic monomers. Monomers based on cationic (meth)acrylic acid esters may be cationized esters of the (meth)acrylic acid containing a quaternized N atom. The cationized esters of the (meth)acrylic acid containing a quaternized N atom can be quaternized dialkylaminoalkyl (meth)acrylates with C1 to C3 in the alkyl and alkylene groups. Suitable cationized esters of the (meth)acrylic acid containing a quaternized N atom can be selected from the group consisting of: ammonium salts of dimethylaminomethyl (meth)acrylate, dimethylaminoethyl (meth)acrylate, dimethylaminopropyl (meth)acrylate, diethylaminomethyl (meth)acrylate, diethylaminoethyl (meth)acrylate; and diethylaminopropyl (meth)acrylate quaternized with methyl chloride. The cationized esters of the (meth)acrylic acid containing a quaternized N atom can be dimethylaminoethyl acrylate, which is quaternized with an alkyl halide, or with methyl chloride or benzyl chloride or dimethyl sulfate (ADAME-Quat). The cationic monomer when based on (meth)acrylamides can be quaternized dialkylaminoalkyl(meth)acrylamides with C1 to C3 in the alkyl and alkylene groups, or dimethylaminopropylacrylamide, which is quaternized with an alkyl halide, or methyl chloride or benzyl chloride or dimethyl sulfate.

Suitable cationic monomer based on a (meth)acrylamide include quaternized dialkylaminoalkyl(meth)acrylamide with C1 to C3 in the alkyl and alkylene groups. The cationic monomer based on a (meth)acrylamide can be dimethylaminopropylacrylamide, which is quaternized with an alkyl halide, especially methyl chloride or benzyl chloride or dimethyl sulfate.

The cationic monomer can be a hydrolysis-stable cationic monomer. Hydrolysis-stable cationic monomers can be, in addition to a dialkylaminoalkyl(meth)acrylamide, all monomers that can be regarded as stable to the OECD hydrolysis test. The cationic monomer can be hydrolysis-stable and the hydrolysis-stable cationic monomer can be selected from the group consisting of: diallyldimethylammonium chloride and water-soluble, cationic styrene derivatives.

The cationic copolymer can be a terpolymer of acrylamide, 2-dimethylammoniumethyl (meth)acrylate quaternized with methyl chloride (ADAME-Q) and 3-dimethylammoniumpropyl(meth)acrylamide quaternized with methyl chloride (DIMAPA-Q). The cationic copolymer can be formed from acrylamide and acrylamidopropyltrimethylammonium chloride, wherein the acrylamidopropyltrimethylammonium chloride has a charge density of from about 1.0 meq/g to about 3.0 meq/g.

The cationic copolymer can have a charge density of from about 1.1 meq/g to about 2.5 meq/g, or from about 1.1 meq/g to about 2.3 meq/g, or from about 1.2 meq/g to about 2.2 meq/g, or from about 1.2 meq/g to about 2.1 meq/g, or from about 1.3 meq/g to about 2.0 meq/g, or from about 1.3 meq/g to about 1.9 meq/g.

The cationic copolymer can have a molecular weight from about 10 thousand g/mol to about 1 million g/mol, or from about 25 thousand g/mol to about 1 million g/mol, or from about 50 thousand g/mol to about 1 million g/mol, or from about 100 thousand g/mol to about 1.0 million g/mol, or from about 150 thousand g/mol to about 1.0 million g/mol.
(a) Cationic Synthetic Polymers

The hair care composition can comprise a cationic synthetic polymer that may be formed from
i) one or more cationic monomer units, and optionally
ii) one or more monomer units bearing a negative charge, and/or
iii) a nonionic monomer,
   wherein the subsequent charge of the copolymer is positive. The ratio of the three types of monomers is given by “m”, “p” and “q” where “m” is the number of cationic monomers, “p” is the number of monomers bearing a negative charge and “q” is the number of nonionic monomers

The cationic polymers can be water soluble or dispersible, non-crosslinked, and synthetic cationic polymers having the following structure:

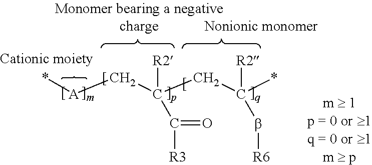

where A, may be one or more of the following cationic moieties:

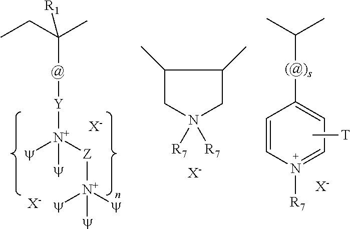

where @=amido, alkylamido, ester, ether, alkyl or alkylaryl;
where Y═C1-C22 alkyl, alkoxy, alkylidene, alkyl or aryloxy;
where i=C1-C22 alkyl, alkyloxy, alkyl aryl or alkyl arylox;
where ψ=C1-C22 alkyl, alkyloxy, aryl or aryloxy;
where R1=H, C1-C4 linear or branched alkyl;
where s=0 or 1, n=0 or 2 1;
where T and R7=C1-C22 alkyl; and
where X—=halogen, hydroxide, alkoxide, sulfate or alkylsulfate.

Where the monomer bearing a negative charge is defined by R2′=H, C1-C4 linear or branched alkyl and R3 as:

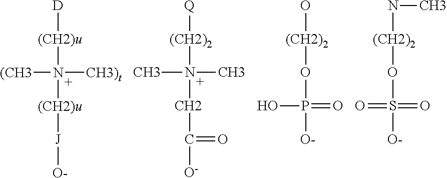

where D=O, N, or S;
where Q=NH₂ or O;
where u=1-6;
where t=0-1; and
where J=oxygenated functional group containing the following elements P, S, C.

Where the nonionic monomer is defined by R2″=H, C1-C4 linear or branched alkyl, R6=linear or branched alkyl, alkyl aryl, aryl oxy, alkyloxy, alkylaryl oxy and β is defined as

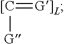

and
where G′ and G″ are, independently of one another, 0, S or N—H and L=0 or 1.

Examples of cationic monomers include aminoalkyl (meth)acrylates, (meth)aminoalkyl (meth)acrylamides; monomers comprising at least one secondary, tertiary or quaternary amine function, or a heterocyclic group containing a nitrogen atom, vinylamine or ethylenimine; diallyldialkyl ammonium salts; their mixtures, their salts, and macromonomers deriving from therefrom.

Further examples of cationic monomers include dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide, ethylenimine, vinylamine, 2-vinylpyridine, 4-vinylpyridine, trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride.

Suitable cationic monomers include those which comprise a quaternary ammonium group of formula —NR₃⁺, wherein R, which is identical or different, represents a hydrogen atom, an alkyl group comprising 1 to 10 carbon atoms, or a benzyl group, optionally carrying a hydroxyl group, and comprise an anion (counter-ion). Examples of anions are halides such as chlorides, bromides, sulphates, hydrosulphates, alkylsulphates (for example comprising 1 to 6 carbon atoms), phosphates, citrates, formates, and acetates.

Suitable cationic monomers include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride.

Additional suitable cationic monomers include trimethyl ammonium propyl (meth)acrylamido chloride.

Examples of monomers bearing a negative charge include alpha ethylenically unsaturated monomers comprising a phosphate or phosphonate group, alpha ethylenically unsaturated monocarboxylic acids, monoalkylesters of alpha ethylenically unsaturated dicarboxylic acids, monoalkylamides of alpha ethylenically unsaturated dicarboxylic acids, alpha ethylenically unsaturated compounds comprising a sulphonic acid group, and salts of alpha ethylenically unsaturated compounds comprising a sulphonic acid group.

Suitable monomers with a negative charge include acrylic acid, methacrylic acid, vinyl sulphonic acid, salts of vinyl sulfonic acid, vinylbenzene sulphonic acid, salts of vinylbenzene sulphonic acid, alpha-acrylamidomethylpropanesulphonic acid, salts of alpha-acrylamidomethylpropanesulphonic acid, 2-sulphoethyl methacrylate, salts of 2-sulphoethyl methacrylate, acrylamido-2-methylpropanesulphonic acid (AMPS), salts of acrylamido-2-methylpropanesulphonic acid, and styrenesulphonate (SS).

Examples of nonionic monomers include vinyl acetate, amides of alpha ethylenically unsaturated carboxylic acids, esters of an alpha ethylenically unsaturated monocarboxylic acids with an hydrogenated or fluorinated alcohol, polyethylene oxide (meth)acrylate (i.e. polyethoxylated (meth)acrylic acid), monoalkylesters of alpha ethylenically unsaturated dicarboxylic acids, monoalkylamides of alpha ethylenically unsaturated dicarboxylic acids, vinyl nitriles, vinylamine amides, vinyl alcohol, vinyl pyrolidone, and vinyl aromatic compounds.

Suitable nonionic monomers include styrene, acrylamide, methacrylamide, acrylonitrile, methylacrylate, ethylacrylate, n-propylacrylate, n-butylacrylate, methylmethacrylate, ethylmethacrylate, n-propylmethacrylate, n-butylmethacrylate, 2-ethyl-hexyl acrylate, 2-ethyl-hexyl methacrylate, 2-hydroxyethylacrylate and 2-hydroxyethylmethacrylate.

The anionic counterion (X—) in association with the synthetic cationic polymers may be any known counterion so long as the polymers remain soluble or dispersible in water, in the hair care composition, or in a coacervate phase of the hair care composition, and so long as the counterions are physically and chemically compatible with the essential components of the hair care composition or do not otherwise unduly impair product performance, stability or aesthetics. Non limiting examples of such counterions include halides (e.g., chlorine, fluorine, bromine, iodine), sulfate and methylsulfate.

The concentration of the cationic polymers ranges about 0.025% to about 5%, from about 0.1% to about 3%, and/or from about 0.2% to about 1%, by weight of the hair care composition.

Suitable cationic cellulose polymers are salts of hydroxyethyl cellulose reacted with trimethyl ammonium substituted epoxide, referred to in the industry (CTFA) as Polyquaternium 10 and available from Dow/Amerchol Corp. (Edison, N.J., USA) in their Polymer LR, JR, and KG series of polymers. Other suitable types of cationic cellulose include the polymeric quaternary ammonium salts of hydroxyethyl cellulose reacted with lauryl dimethyl ammonium-substituted epoxide referred to in the industry (CTFA) as Polyquaternium 24. These materials are available from Dow/Amerchol Corp. under the tradename Polymer LM-200. Other suitable types of cationic cellulose include the polymeric quaternary ammonium salts of hydroxyethyl cellulose reacted with lauryl dimethyl ammonium-substituted epoxide and trimethyl ammonium substituted epoxide referred to in the industry (CTFA) as Polyquaternium 67. These materials are available from Dow/Amerchol Corp. under the tradename SoftCAT Polymer SL-5, SoftCAT Polymer SL-30, Polymer SL-60, Polymer SL-100, Polymer SK-L, Polymer SK-M, Polymer SK-MH, and Polymer SK-H.

E. Conditioning Agents

The hair care compositions may comprise one or more conditioning agent. Conditioning agents include materials that are used to give a particular conditioning benefit to hair and/or skin. The conditioning agents useful in the hair care compositions of the present invention typically comprise a water-insoluble, water-dispersible, non-volatile, liquid that forms emulsified, liquid particles. Suitable conditioning agents for use in the hair care composition are those conditioning agents characterized generally as silicones (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix.

### 1. Silicone Conditioning Agents

The hair care composition can comprise from about 0% to about 20% by weight, alternatively from about 6% to about 18% by weight; and alternatively from about 8% to about 16% by weight of one of more silicones with a particle size of less than about 300 nm, alternatively less than about 200 nm, and alternatively less than about 100 nm. The silicone can be in the form of a nanoemulsion.

The particle size of the one or more silicones may be measured by dynamic light scattering (DLS). A Malvern Zetasizer Nano ZEN3600 system (www.malvern.com) using He—Ne laser 633 nm may be used for the measurement at 25° C.

The autocorrelation function may be analyzed using the Zetasizer Software provided by Malvern Instruments, which determines the effective hydrodynamic radius, using the Stokes-Einstein equation:

$$D = \frac{{k}_{B} T}{6 πη R}$$

wherein k_{B} is the Boltzmann Constant, T is the absolute temperature, η is the viscosity of the medium, D is the mean diffusion coefficient of the scattering species, and R is the hydrodynamic radius of particles.

Particle size (i.e. hydrodynamic radius) may be obtained by correlating the observed speckle pattern that arises due to Brownian motion and solving the Stokes-Einstein equation, which relates the particle size to the measured diffusion constant, as is known in the art.

For each sample, 3 measurements may be made and Z-average values may be reported as the particle size.

The one or more silicones may be in the form of a nanoemulsion. The nanoemulsion may comprise any silicone suitable for application to the skin and/or hair.

The one or more silicones may include in their molecular structure polar functional groups such as Si—OH (present in dimethiconols), primary amines, secondary amines, tertiary amines, and quaternary ammonium salts. The one or more silicones may be selected from the group consisting of aminosilicones, pendant quaternary ammonium silicones, terminal quaternary ammonium silicones, amino polyalkylene oxide silicones, quaternary ammonium polyalkylene oxide silicones, and amino morpholino silicones.

The one or more silicones may comprise:

(a) at least one aminosilicone corresponding to formula (V):

R′ₐG₃₋ₐ-Si(OSiG₂)ₙ-(OSiG_{b}R′_{2-b})ₘ—O-SiG₃₋ₐR′ₐ (I)

in which:

G is chosen from a hydrogen atom, a phenyl group, OH group, and C₁-C₈alkyl groups, for example methyl,
a is an integer ranging from 0 to 3, and in one embodiment a is 0,
b is chosen from 0 and 1, and in one embodiment b is 1,
m and n are numbers such that the sum (n+m) can range for example from 1 to 2 000, such as for example from 50 to 150, wherein n can be for example chosen from numbers ranging from 0 to 1 999, such as for example from 49 to 149, and wherein m can be chosen from numbers ranging for example from 1 to 2 000, such as for example from 1 to 10;
R′ is a monovalent group of formula —C_{q}H_{2q}L in which q is a number from 2 to 8 and L is an optionally quaternized amine group chosen from the groups:
—NR″—CH₂—CH₂—N′(R¹)₂,

### —N(R″)₂,

—N⁺(R″)₃A⁻,
—N⁺H(R″)₂A⁻,
—N⁺H₂(R″)A⁻, and
—N(R″)—CH₂—CH₂—N⁺R″H₂A⁻,
in which R″ can be chosen from a hydrogen atom, phenyl groups, benzyl groups, and saturated monovalent hydrocarbon-based groups, such as for example an alkyl group comprising from 1 to 20 carbon atoms, and A⁻ is chosen from halide ions such as, for example, fluoride, chloride, bromide and iodide.

The one or more silicones may include those corresponding to formula (1) wherein a=0, G=methyl, m and n are numbers such that the sum (n+m) can range for example from 1 to 2 000, such as for example from 50 to 150, wherein n can be for example chosen from numbers ranging from 0 to 1 999, such as for example from 49 to 149, and wherein m can be chosen from numbers ranging for example from 1 to 2 000, such as for example from 1 to 10; and L is —N(CH₃)₂ or —NH₂, alternatively —NH₂.

Additional said at least one aminosilicone of the invention include:
(b) pendant quaternary ammonium silicones of formula (VII):

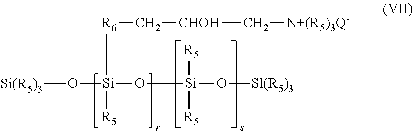

in which:
R₅ is chosen from monovalent hydrocarbon-based groups comprising from 1 to 18 carbon atoms, such as C₁-C₁₈ alkyl groups and C₂-C₁₈ alkenyl groups, for example methyl;
R₆ is chosen from divalent hydrocarbon-based groups, such as divalent C₁-C₁₈ alkylene groups and divalent C₁-C₁₈ alkylenoxy groups, for example C₁-C₅ alkylenoxy groups, wherein said R₆ is bonded to the Si by way of an SiC bond;
Q⁻ is an anion that can be for example chosen from halide ions, such as chloride, and organic acid salts (such as acetate);
r is an average statistical value ranging from 2 to 20, such as from 2 to 8;
s is an average statistical value ranging from 20 to 200, such as from 20 to 50.

Such aminosilicones are described more particularly in U.S. Pat. No. 4,185,087, the disclosure of which is incorporated by reference herein.

A silicone which falls within this class is the silicone sold by the company Union Carbide under the name “Ucar Silicone ALE 56”.

Further examples of said at least one aminosilicone include:

c) quaternary ammonium silicones of formula (VIIb):

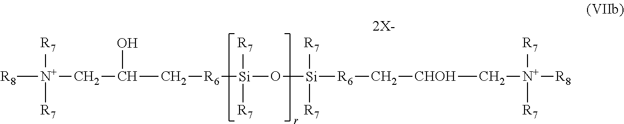

which:
groups R₇, which may be identical or different, are each chosen from monovalent hydrocarbon-based groups comprising from 1 to 18 carbon atoms, such as C₁-C₁₈ alkyl groups, for example methyl, C₂-C₁₈ alkenyl groups, and rings comprising 5 or 6 carbon atoms;
R₆ is chosen from divalent hydrocarbon-based groups, such as divalent C₁-C₁₈ alkylene groups and divalent C₁-C₁₈alkylenoxy, for example C₁-C₈, group connected to the Si by an SiC bond;
R₈, which may be identical or different, represent a hydrogen atom, a monovalent hydrocarbon-based group comprising from 1 to 18 carbon atoms, and in particular a C₁-C₁₈ alkyl group, a C₂-C₁₈ alkenyl group or a group —R₆—NHCOR₇;
X⁻ is an anion such as a halide ion, in particular chloride, or an organic acid salt (acetate, etc.);
r represents an average statistical value from 2 to 200 and in particular from 5 to 100.
Such silicones are described, for example, in application EP-A-0 530 974, the disclosure of which is incorporated by reference herein.
Silicones falling within this class are the silicones sold by the company Eovnik under the names Abil Quat 3270, Abil Quat 3272, Abil Quat 3474 and Abil ME 45.
Further examples of said at least one aminosilicone include:
d) quaternary ammonium and polyalkylene oxide silicones
wherein the quaternary nitrogen groups are located in the polysiloxane backbone, at the termini, or both.
Such silicones are described in PCT Publication No. WO 2002/010257, the disclosure of which is incorporated by reference herein.
Silicones falling within this class are the silicones sold by the company Momentive under the names Silsoft Q.
(e) Aminofunctional silicones having morpholino groups of formula (V):

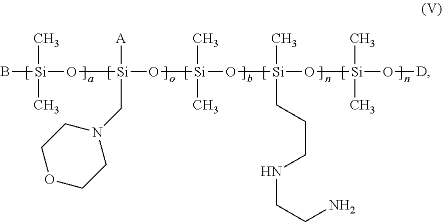

in which

A denotes a structural unit (I), (II), or (III) bound via —O—

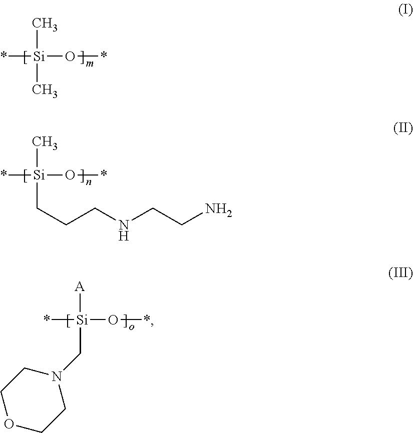

or an oligomeric or polymeric residue, bound via —O—, containing structural units of formulas (I), (II), or (III), or half of a connecting oxygen atom to a structural unit (III), or denotes —OH,
denotes a bond to one of the structural units (I), (II), or (III), or denotes a terminal group B (Si-bound) or D (O-bound),
B denotes an —OH, —O—Si(CH₃)₃, —O—Si(CH₃)₂₀H, —O—Si(CH₃)₂OCH₃ group,
D denotes an —H, —Si(CH₃)₃, —Si(CH₃)₂₀H, —Si(CH₃)₂OCH₃ group,
a, b, and c denote integers between 0 and 1000, with the provision that a+b+c>0,
m, n, and o denote integers between 1 and 1000.

Aminofunctional silicones of this kind bear the INCI name: Amodimethicone/Morpholinomethyl Silsesquioxane Copolymer. A particularly suitable amodimethicone is the product having the commercial name Wacker Belsil® ADM 8301E.

Examples of such silicones are available from the following suppliers: offered by the company Dow Corning:Fluids: 2-8566, AP 6087, AP 6088, DC 8040 Fluid, fluid 8822A DC, DC 8803 & 8813 polymer, 7-6030, AP-8104, AP 8201; Emulsions: CE-8170 AF Micro Emulsion, 2-8177, 2-8194 Microemulsion, 9224 Emulsion, 939, 949, 959, DC 5-7113 Quat Microemulsion, DC 5-7070 Emulsion, DC CE-8810, CE 8401 Emulsion, CE 1619, Dow Corning Toray SS-3551, Dow Corning Toray SS-3552;

offered by the company Wacker:Wacker Belsil ADM 652, ADM 656, 1100, 1600, 1650 (fluids) ADM 6060 (linear amodimethicone) emulsion; ADM 6057 E (branched amodimethicone) emulsion; ADM 8020 VP (micro emulsion); SLM 28040 (micro emulsion);
offered by the Company Momentive:Silsoft 331, SF1708, SME 253 & 254 (emulsion), SM2125 (emulsion), SM 2658 (emulsion), Silsoft Q (emulsion)
offered by the company Shin-Etsu:KF-889, KF-867S, KF-8004, X-52-2265 (emulsion);
offered by the Company Siltech Silicones:Siltech E-2145, E-Siltech 2145-35;
offered by the company Evonik Industries:Abil T Quat 60th

Some non-limiting examples of aminosilicones include the compounds having the following INCI names: Silicone Quaternium-1, Silicone Quaternium-2, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, Silicone Quaternium-16, Silicone Quaternium-17, Silicone Quaternium-18, Silicone Quaternium-20, Silicone Quaternium-21, Silicone Quaternium-22, Quaternium-80, as well as Silicone Quaternium-2 Panthenol Succinate and Silicone Quaternium-16/Glycidyl Dimethicone Crosspolymer.

The aminosilicones can be supplied in the form of a nanoemulsion and include MEM 9049, MEM 8177, MEM 0959, MEM 8194, SME 253, and Silsoft Q.

The one or more silicones may include dimethicones, and/or dimethiconols. The dimethiconols are hydroxyl terminated dimethylsilicones represented by the general chemical formulas

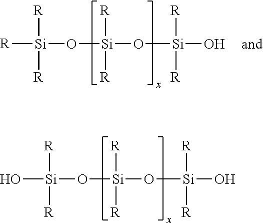

wherein R is an alkyl group (preferably R is methyl or ethyl, more preferably methyl) and x is an integer up to about 500, chosen to achieve the desired molecular weight. Commercial dimethiconols typically are sold as mixtures with dimethicone or cyclomethicone (e.g., Dow Corning® 1401, 1402, and 1403 fluids).

### 2. Non-Silicone Conditioning Agents

The conditioning agent of the hair care compositions described herein may also comprise at least one organic conditioning agents, either alone or in combination with other conditioning agents, such as the silicones described above. Non-limiting examples of organic conditioning agents are described below.

a. Hydrocarbon Oils

Suitable organic conditioning agents for use as conditioning agents in hair care compositions include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils can be from about C₁₂ to about C₁₉. Branched chain hydrocarbon oils, including hydrocarbon polymers, typically will contain more than 19 carbon atoms.

b. Polyolefins

Organic conditioning oils for use in the hair care compositions described herein also include liquid polyolefins, including liquid poly-α-olefins and/or hydrogenated liquid poly-α-olefins. Polyolefins for use herein can be prepared by polymerization of C₄ to about C₁₄ olefenic monomers, alternatively from about C₆ to about C₁₂.

c. Fatty Esters

Other suitable organic conditioning agents for use as a conditioning agent in the hair care compositions described herein include fatty esters having at least 10 carbon atoms. These fatty esters include esters with hydrocarbyl chains derived from fatty acids or alcohols. The hydrocarbyl radicals of the fatty esters hereof may include or have covalently bonded thereto other compatible functionalities, such as amides and alkoxy moieties (e.g., ethoxy or ether linkages, etc.). Other oligomeric or polymeric esters, prepared from unsaturated glyceryl esters can also be used as conditioning materials.

d. Fluorinated Conditioning Compounds

Fluorinated compounds suitable for delivering conditioning to hair as organic conditioning agents include perfluoropolyethers, perfluorinated olefins, fluorine based specialty polymers that may be in a fluid or elastomer form similar to the silicone fluids previously described, and perfluorinated dimethicones.

e. Fatty Alcohols
Other suitable organic conditioning oils for use in the hair care compositions described herein include, but are not limited to, fatty alcohols having at least about 10 carbon atoms, about 10 to about 22 carbon atoms, alternatively about 12 to about 16 carbon atoms.
f. Alkyl Glucosides and Alkyl Glucoside Derivatives
Suitable organic conditioning oils for use in the hair care compositions described herein include, but are not limited to, alkyl glucosides and alkyl glucoside derivatives. Specific non-limiting examples of suitable alkyl glucosides and alkyl glucoside derivatives include Glucam E-10, Glucam E-20, Glucam P-10, and Glucquat 125 commercially available from Amerchol.
g. Polyethylene Glycols

Additional compounds useful herein as conditioning agents include polyethylene glycols and polypropylene glycols having a molecular weight of up to about 2,000,000 such as those with CTFA names PEG-200, PEG-400, PEG-600, PEG-1000, PEG-2M, PEG-7M, PEG-14M, PEG-45M and mixtures thereof.

F. Aerosol Foam Dispenser

The aerosol foam dispenser may comprise a reservoir for holding the hair care composition. The reservoir may be made out of any suitable material selected from the group consisting of plastic, metal, alloy, laminate, and combinations thereof. The reservoir may be for one-time use. The reservoir may be removable from the aerosol foam dispenser. Alternatively, the reservoir may be integrated with the aerosol foam dispenser. There may be two or more reservoirs.

The reservoir may be comprised of a material selected from the group consisting of rigid materials, flexible materials, and combinations thereof. The reservoir may be comprised of a rigid material if it does not collapse under external atmospheric pressure when it is subject to an interior partial vacuum.

G. Foaming Agent

The hair care composition described herein may comprise from about from about 3% to about 12% propellant or foaming agent, alternatively from about 3% to about 10% propellant or foaming agent, and alternatively from about 5% to about 8% propellant or foaming agent, by weight of the hair care composition.

The present invention pertains to the use of trans-1,3,3,3-tetrafluoropropene (“HFO”) (Solstice® Propellant HFO-1234ze available by Honeywell) as a foaming agent within shampoo formulations.

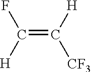

When used as a foaming agent, trans-1,3,3,3-tetrafluoropropene has been found to have unique advantages over the use of low vapor pressure hydrocarbon foaming agents (such as commonly used A46 which is a mixture of 84.8% isobutane and 15.2% propane) in that it enables significantly higher foam densities (approximately 2× greater) versus hydrobarbon propellants and at equal formula pressure and formula % saturated pressure. The higher density enables higher gravimetric foam dosage per unit volume of the resulting dispensed foam shampoo and making it easier to achieve sufficient dosage from a low density foam shampoo form relative to a high density liquid shampoo form. The pressure and % saturated pressure is important to enable sufficient foam dispensing over the life of the product (from beginning to middle to end of the pressurized container). The trans-1,3,3,3-tetrafluoropropene has been found to result in gloss or shine of the dispensed foam.

H. Viscosity

The hair care composition may have a liquid phase viscosity of less than about 3000 centipoise (cP), alternatively from about 1 cP centipoise to about 3000 cP, alternatively from about 2 cP centipoise to about 3000 cP, alternatively from about 10 cP to about 2500 cP, alternatively from about 20 to about 2500 cP, alternatively from about 50 to about 2000 cP measured at 26.5° C. as defined herein. The viscosities are measured by a Cone and Plate Controlled Stress Brookfield Rheometer R/S Plus, by Brookfield Engineering Laboratories, Stoughton, Mass. The cone used (Spindle C-75-1) has a diameter of 75 mm and 10 angle. The viscosity is determined using a steady state flow experiment at constant shear rate of 2 s⁻¹ at a temperature of 26.5° C. The sample size is 2.5 ml and the total measurement reading time is 3 minutes.

I. Foam Density

Foam density is measured by placing a 100 ml beaker onto a mass balance, tarring the mass of the beaker and then dispensing product from the aerosol container into the 100 ml beaker until the volume of the foam is above the rim of the vessel. The foam is made level with the top of the beaker by immediately scraping a spatula across it. The resulting mass of the 100 ml of foam is then divided by the volume (100) to determine the foam density in units of g/ml.

The foam density of the foam dispensed by the aerosol package is between about 0.03 to about 0.35 g/ml. The foam can also have a foam density of from about 0.05 g/cm3 to about 0.35 g/cm3, alternatively from about 0.08 g/cm3 to about 0.25 g/cm3, alternatively from about 0.08 g/cm3 to about 0.2 g/cm3, alternatively from about 0.08 g/cm3 to about 0.18 g/cm3, alternatively from about 0.08 g/cm3 to about 0.15 g/cm3, alternatively from about 0.08 g/cm3 to about 0.12 g/cm3; alternatively from about 0.1 g/cm3 to about 0.12 g/cm3, alternatively from about 0.12 g/cm3 to about 0.2 g/cm3, or alternatively from about 0.15 g/cm3 to about 0.2 g/cm3.

J. Perfume

The hair care composition may comprise from about 0.5% to about 7%, alternatively from about 1% to about 6%, and alternatively from about 2% to about 5% perfume, by weight of the hair care composition.

The hair care composition may have a silicone to perfume ratio of from about 95:5 to about 50:50, alternatively from about 90:10 to about 60:40, and alternatively from about 85:15 to about 70:30.

Examples of suitable perfumes may be provided in the CTFA (Cosmetic, Toiletry and Fragrance Association) 1992 International Buyers Guide, published by CFTA Publications and OPD 1993 Chemicals Buyers Directory 80th Annual Edition, published by Schnell Publishing Co. A plurality of perfume components may be present in the hair care composition.

K. Optional Ingredients

The hair conditioning composition described herein may optionally comprise one or more additional components known for use in hair care or personal care products, provided that the additional components are physically and chemically compatible with the essential components described herein, or do not otherwise unduly impair product stability, aesthetics or performance. Such optional ingredients are most typically those materials approved for use in cosmetics and that are described in reference books such as the CTFA Cosmetic Ingredient Handbook, Second Edition, The Cosmetic, Toiletries, and Fragrance Association, Inc. 1988, 1992. Individual concentrations of such additional components may range from about 0.001 wt % to about 10 wt % by weight of the conditioning composition.

Emulsifiers suitable as an optional ingredient herein include mono- and di-glycerides, fatty alcohols, polyglycerol esters, propylene glycol esters, sorbitan esters and other emulsifiers known or otherwise commonly used to stabilized air interfaces, as for example those used during preparation of aerated foodstuffs such as cakes and other baked goods and confectionary products, or the stabilization of cosmetics such as hair mousses.

Further non-limiting examples of such optional ingredients include preservatives, perfumes or fragrances, cationic polymers, viscosity modifiers, coloring agents or dyes, conditioning agents, hair bleaching agents, thickeners, moisturizers, foam boosters, additional surfactants or nonionic cosurfactants, emollients, pharmaceutical actives, vitamins or nutrients, sunscreens, deodorants, sensates, plant extracts, nutrients, astringents, cosmetic particles, absorbent particles, adhesive particles, hair fixatives, fibers, reactive agents, skin lightening agents, skin tanning agents, anti-dandruff agents, perfumes, exfoliating agents, acids, bases, humectants, enzymes, suspending agents, pH modifiers, hair colorants, hair perming agents, pigment particles, anti-acne agents, anti-microbial agents, sunscreens, tanning agents, exfoliation particles, hair growth or restorer agents, insect repellents, shaving lotion agents, non-volatile solvents or diluents (water-soluble and water-insoluble), co-solvents or other additional solvents, and similar other materials.

Non-limiting examples of anti-dandruff agents include one material or a mixture selected from the groups consisting of: azoles, such as climbazole, ketoconazole, itraconazole, econazole, and elubiol; hydroxyl pyridones, such as octopirox (piroctone olamine), ciclopirox, rilopirox, and MEA-Hydroxyoctyloxypyridinone; kerolytic agents, such as salicylic acid and other hydroxy acids; strobilurins such as azoxystrobin and metal chelators such as 1,10-phenanthroline. In another embodiment, the azole anti-microbials is an imidazole selected from the group consisting of: benzimidazole, benzothiazole, bifonazole, butaconazole nitrate, climbazole, clotrimazole, croconazole, eberconazole, econazole, elubiol, fenticonazole, fluconazole, 10 flutimazole, isoconazole, ketoconazole, lanoconazole, metronidazole, miconazole, neticonazole, omoconazole, oxiconazole nitrate, sertaconazole, sulconazole nitrate, tioconazole, thiazole, and mixtures thereof, or the azole anti-microbials is a triazole selected from the group consisting of: terconazole, itraconazole, and mixtures thereof. In an embodiment, the azole anti-microbial active is ketoconazole. In an embodiment, the sole antimicrobial active is ketoconazole.

### Method of Treating Hair

The method of treating the hair described herein comprises (1) providing a hair care composition, as described herein, (2) dispensing the hair care composition as a liquid form or a foam form using a mechanical foam dispenser or an aerosol foam dispenser; (3) applying the composition to the hair; and (4) rinsing the composition from the hair. The hair care composition can form a stable foam. A foam is stable when it substantially sustains its volume from the time of dispensing to its application on hair. The foam can have a density of from about 0.025 g/cm³ to about 0.15 g/cm³ when dispensed from the aerosol foam dispenser

### EXAMPLES

The following examples illustrate the hair care composition described herein. The exemplified compositions can be prepared by conventional formulation and mixing techniques. It will be appreciated that other modifications of the present invention within the skill of those in the shampoo formulation art can be undertaken without departing from the spirit and scope of this invention. All parts, percentages, and ratios herein are by weight unless otherwise specified. Some components may come from suppliers as dilute solutions. The amount stated reflects the weight percent of the active material, unless otherwise specified.

The following are non-limiting examples of the hair care composition described herein.

### Examples and Results

### Shampoo Compositions Comprising HFO

| | Ex. 1 | Ex. 2 | Ex. 3 | Ex. 4 | Ex. 5 |
|---|---|---|---|---|---|
| Sodium laureth-1-sulfate | | | | | |
| SLE1S¹ | | | | | |
| Branched sodium trideceth-2- | | | | | |
| sulfate ST2S² | | | | | |
| Sodium Undecyl Sulfate (C11 | 24 | 24 | 24 | 24 | 24 |
| 70% active)³ | | | | | |
| Cocoamidopropyl betaine⁴ | | | | | |
| Sodium Lauroamphoacetate⁵ | | | | | |
| Lauramidopropyl Betaine | 6 | 6 | 6 | 6 | 6 |
| (LAPB 35% active)⁶ | | | | | |
| Dipropylene glycol | 4 | 4 | 4 | 4 | 4 |
| Guar, Hydroxylpropyl | 0.40 | 0.40 | 0.40 | 0.40 | 0.40 |
| Trimonium Chloride, Jaguar | | | | | |
| C-500⁷ | | | | | |
| Guar, Hydroxylpropyl | | | | | |
| Trimonium Chloride, N- | | | | | |
| Hance 3196⁸ | | | | | |
| Silicone quaternium⁹ | | | | | |
| Perfume | 2.40 | 2.40 | 2.40 | 2.40 | 2.40 |
| HFO¹⁰ | 3.1 | 4.0 | 5.2 | 6.0 | 6.9 |
| Preservatives, pH adjusters | Up to 1% | Up to 1% | Up to 1% | Up to 1% | Up to 1% |
| Water | Q.S. | Q.S. | Q.S. | Q.S. | Q.S. |
| Total anionic Surfactant | 24.00 | 24.00 | 24.00 | 24.00 | 24.00 |
| Total surfactant | 30.00 | 30.00 | 30.00 | 30.00 | 30.00 |
| HFO/DPG | 0.76 | 0.99 | 1.30 | 1.51 | 1.74 |
| Bulk Viscosity (cps) | 556 | 556 | 556 | 556 | 556 |
| Foam Density (g/ml) | 0.25 | 0.18 | 0.14 | 0.11 | 0.10 |
| Stability | stable | stable | stable | stable | stable |
| | Ex. 6 | Ex. 7 | Ex. 8 | Ex. 9 | Ex. 10 |
| Sodium laureth-1-sulfate | | | | | |
| SLE1S¹ | | | | | |
| Branched sodium trideceth-2- | | | | | |
| sulfate ST2S² | | | | | |
| Sodium Undecyl Sulfate (C11 | 24 | 24 | 24 | 24 | 24 |
| 70% active)³ | | | | | |
| Cocoamidopropyl betaine⁴ | | | | | |
| Sodium Lauroamphoacetate⁵ | | | | | |
| Lauramidopropyl Betaine | 6 | 6 | 6 | 6 | 6 |
| (LAPB 35% active)⁶ | | | | | |
| Dipropylene glycol | 4 | 4 | 4 | 4 | 4 |
| Guar, Hydroxylpropyl | 0.40 | 0.40 | 0.40 | 0.40 | 0.40 |
| Trimonium Chloride, Jaguar | | | | | |
| C-500⁷ | | | | | |
| Guar, Hydroxylpropyl | | | | | |
| Trimonium Chloride, N- | | | | | |
| Hance 3196⁸ | | | | | |
| Silicone quaternium⁹ | | | | | |
| Perfume | 2.40 | 2.40 | 2.40 | 2.40 | 2.40 |
| HFO¹⁰ | 7.8 | 8.5 | 9.3 | 10.6 | 11.4 |
| Preservatives, pH adjusters | Up to 1% | Up to 1% | Up to 1% | Up to 1% | Up to 1% |
| Water | Q.S. | Q.S. | Q.S. | Q.S. | Q.S. |
| Total anionic Surfactant | 24.00 | 24.00 | 24.00 | 24.00 | 24.00 |
| Total surfactant | 30.00 | 30.00 | 30.00 | 30.00 | 30.00 |
| HFO/DPG | 1.95 | 2.13 | 2.33 | 2.66 | 2.86 |
| Bulk Viscosity (cps) | 556 | 556 | 556 | 556 | 556 |
| Foam Density (g/ml) | 0.09 | 0.07 | 0.06 | 0.06 | 0.05 |
| Stability | stable | stable | stable | stable | stable |
| | Ex. 11 | Ex. 12 | Ex. 13 | Ex. 14 | Ex. 15 |
| Sodium laureth-1-sulfate | | | 18 | 18 | 18 |
| SLE1S¹ | | | | | |
| Branched sodium trideceth-2- | | | 8 | 8 | 8 |
| sulfate ST2S² | | | | | |
| Sodium Undecyl Sulfate (C11 | 12 | 12 | | | |
| 70% active)³ | | | | | |
| Cocoamidopropyl betaine⁴ | | | 2 | 2 | 2 |
| Sodium Lauroamphoacetate⁵ | | | 2 | 2 | 2 |
| Lauramidopropyl Betaine | 3 | 3 | | | |
| (LAPB 35% active)⁶ | | | | | |
| Dipropylene glycol | 2 | 2 | 4 | 4 | 4 |
| Guar, Hydroxylpropyl | 0.20 | 0.20 | 0.40 | 0.40 | 0.40 |
| Trimonium Chloride, Jaguar | | | | | |
| C-500⁷ | | | | | |
| Guar, Hydroxylpropyl | | | | | |
| Trimonium Chloride, N- | | | | | |
| Hance 3196⁸ | | | | | |
| Silicone quaternium⁹ | | | | | |
| Perfume | 1.20 | 1.20 | 2.40 | 2.40 | 2.40 |
| HFO¹⁰ | 4.2 | 5.4 | 3.1 | 4.2 | 4.9 |
| Preservatives, pH adjusters | Up to 1% | Up to 1% | Up to 1% | Up to 1% | Up to 1% |
| Water | Q.S. | Q.S. | Q.S. | Q.S. | Q.S. |
| Total anionic Surfactant | 12.00 | 12.00 | 26.00 | 26.00 | 26.00 |
| Total surfactant | 15.00 | 15.00 | 30.00 | 30.00 | 30.00 |
| HFO/DPG | 2.08 | 2.72 | 0.79 | 1.04 | 1.23 |
| Bulk Viscosity (cps) | 50 | 50 | 2029 | 2029 | 2029 |
| Foam Density (g/ml) | 0.14 | 0.11 | 0.28 | 0.20 | 0.16 |
| Stability | stable | stable | stable | stable | stable |
| | Ex. 16 | Ex. 17 | Ex. 18 | Ex. 19 | Ex. 20 |
| Sodium laureth-1-sulfate | 18 | 18 | 18 | 18 | 18 |
| SLE1S¹ | | | | | |
| Branched sodium trideceth-2- | 8 | 8 | 8 | 8 | 8 |
| sulfate ST2S² | | | | | |
| Sodium Undecyl Sulfate (C11 | | | | | |
| 70% active)³ | | | | | |
| Cocoamidopropyl betaine⁴ | 2 | 2 | 2 | 2 | 2 |
| Sodium Lauroamphoacetate⁵ | 2 | 2 | 2 | 2 | 2 |
| Lauramidopropyl Betaine | | | | | |
| (LAPB 35% active)⁶ | | | | | |
| Dipropylene glycol | 4 | 4 | 4 | 4 | 4 |
| Guar, Hydroxylpropyl | | 0.40 | 0.40 | 0.40 | 0.40 |
| Trimonium Chloride, Jaguar | | | | | |
| C-500⁷ | | | | | |
| Guar, Hydroxylpropyl | | | | | |
| Trimonium Chloride, N- | | | | | |
| Hance 3196⁸ | | | | | |
| Silicone quaternium⁹ | | | | | |
| Perfume | 2.40 | 2.40 | 2.40 | 2.40 | 2.40 |
| HFO¹⁰ | 5.2 | 5.8 | 6.7 | 8.0 | 9.5 |
| Preservatives, pH adjusters | Up to 1% | Up to 1% | Up to 1% | Up to 1% | Up to 1% |
| Water | Q.S. | Q.S. | Q.S. | Q.S. | Q.S. |
| Total anionic Surfactant | 26.00 | 26.00 | 26.00 | 26.00 | 26.00 |
| Total surfactant | 30.00 | 30.00 | 30.00 | 30.00 | 30.00 |
| HFO/DPG | 1.30 | 1.46 | 1.67 | 2.00 | 2.37 |
| Bulk Viscosity (cps) | 1275 | 2029 | 2029 | 2029 | 2029 |
| Foam Density(g/ml) | 0.20 | 0.13 | 0.11 | 0.10 | 0.08 |
| Stability | stable | stable | stable | stable | stable |
| | Ex. 21 | Ex. 22 | Ex. 23 | Ex. 24 | Ex. 25 |
| Sodium laureth-1-sulfate | 18 | 18 | 18 | 18 | 13 |
| SLE1S¹ | | | | | |
| Branched sodium trideceth-2- | 8 | 8 | 8 | 8 | 13 |
| sulfate ST2S² | | | | | |
| Sodium Undecyl Sulfate (C11 | | | | | |
| 70% active)³ | | | | | |
| Cocoamidopropyl betaine⁴ | 2 | 2 | 2 | 4 | 4 |
| Sodium Lauroamphoacetate⁵ | 2 | 2 | 2 | | |
| Lauramidopropyl Betaine | | | | | |
| (LAPB 35% active)⁶ | | | | | |
| Dipropylene glycol | 8 | 8 | 4 | 4 | 4 |
| Guar, Hydroxylpropyl | 0.40 | 0.40 | 0.30 | | |
| Trimonium Chloride, Jaguar | | | | | |
| C-500⁷ | | | | | |
| Guar, Hydroxylpropyl | | | 0.10 | | |
| Trimonium Chloride, N- | | | | | |
| Hance 3196⁸ | | | | | |
| Silicone quaternium⁹ | | | 1.50 | | |
| Perfume | 2.40 | 2.40 | 2.40 | 2.40 | 2.40 |
| HFO¹⁰ | 5.2 | 7.8 | 7.8 | 5.2 | 5.2 |
| Preservatives, pH adjusters | Up to 1% | Up to 1% | Up to 1% | Up to 1% | Up to 1% |
| Water | Q.S. | Q.S. | Q.S. | Q.S. | Q.S. |
| Total anionic Surfactant | 26.00 | 26.00 | 26.00 | 26.00 | 26.00 |
| Total surfactant | 30.00 | 30.00 | 30.00 | 30.00 | 30.00 |
| HFO/DPG | 0.65 | 0.98 | 1.96 | 1.30 | 1.30 |
| Bulk Viscosity (cps) | 930 | 930 | 1406 | 2324 | 961 |
| Foam Density (g/ml) | 0.19 | 0.12 | 0.07 | 0.20 | 0.20 |
| Stability | stable | stable | stable | stable | stable |
| | Ex. 26 | Ex. 27 | Ex. 28 | Ex. 29 | Ex. 30 |
| Sodium laureth-1-sulfate | 22 | 16.45 | 19.27 | 27.00 | 25.00 |
| SLE1S¹ | | | | | |
| Branched sodium trideceth-2- | 4 | 10.47 | 1.04 | 0.00 | 0.00 |
| sulfate ST2S² | | | | | |
| Sodium Undecyl Sulfate (C11 | | | | | |
| 70% active)³ | | | | | |
| Cocoamidopropyl betaine⁴ | 2 | 1.54 | 8.66 | 0.00 | 5.00 |
| Sodium Lauroamphoacetate⁵ | 2 | 1.88 | 1.96 | 3.00 | 0.00 |
| Lauramidopropyl Betaine | | | | | |
| (LAPB 35% active)⁶ | | | | | |
| Dipropylene glycol | 4 | 5.47 | 7.78 | 8 | 8 |
| Guar, Hydroxylpropyl | | | | | |
| Trimonium Chloride, Jaguar | | | | | |
| C-500⁷ | | | | | |
| Guar, Hydroxylpropyl | | | | | |
| Trimonium Chloride, N- | | | | | |
| Hance 3196⁸ | | | | | |
| Silicone quaternium⁹ | | | | | |
| Perfume | 2.40 | 2.14 | 2.27 | 2.40 | 1.60 |
| HFO¹⁰ | 5.2 | 5.2 | 5.2 | 5.2 | 5.2 |
| Preservatives, pH adjusters | Up to 1% | Up to 1% | Up to 1% | Up to 1% | Up to 1% |
| Water | Q.S. | Q.S. | Q.S. | Q.S. | Q.S. |
| Total anionic Surfactant | 26.00 | 26.92 | 20.31 | 27.00 | 25.00 |
| Total surfactant | 30.00 | 30.34 | 30.94 | 30.00 | 30.00 |
| HFO/DPG | 1.30 | 0.95 | 0.67 | 0.65 | 0.65 |
| Bulk Viscosity (cps) | 2752 | 724 | 1084 | 539 | 1289 |
| Foam Density (g/ml) | 0.20 | 0.15 | 0.16 | 0.14 | 0.15 |
| Stability | stable | stable | stable | stable | stable |
| | Ex. 31 | Ex. 32 | Ex. 33 | Ex. 34 | Ex. 35 |
| Sodium laureth-1-sulfate | 20.00 | 15.00 | 17.45 | 22.01 | 3.64 |
| SLE1S¹ | | | | | |
| Branched sodium trideceth-2- | 0.00 | 15.00 | 8.33 | 3.39 | 25.61 |
| sulfate ST2S² | | | | | |
| Sodium Undecyl Sulfate (C11 | | | | | |
| 70% active)³ | | | | | |
| Cocoamidopropyl betaine⁴ | 4.00 | 0.00 | 9.26 | 3.11 | 0.28 |
| Sodium Lauroamphoacetate⁵ | 6.00 | 0.00 | 0.07 | 2.46 | 1.41 |
| Lauramidopropyl Betaine | | | | | |
| (LAPB 35% active)⁶ | | | | | |
| Dipropylene glycol | 8 | 8 | 9.48 | 9.71 | 9.87 |
| Guar, Hydroxylpropyl | | | | | |
| Trimonium Chloride, Jaguar | | | | | |
| C-500⁷ | | | | | |
| Guar, Hydroxylpropyl | | | | | |
| Trimonium Chloride, N- | | | | | |
| Hance 3196⁸ | | | | | |
| Silicone quaternium⁹ | | | | | |
| Perfume | 1.60 | 1.60 | 1.88 | 2.07 | 2.31 |
| HFO¹⁰ | 5.2 | 5.2 | 5.2 | 5.2 | 5.2 |
| Preservatives, pH adjusters | Up to 1% | Up to 1% | Up to 1% | Up to 1% | Up to 1% |
| Water | Q.S. | Q.S. | Q.S. | Q.S. | Q.S. |
| Total anionic Surfactant | 20.00 | 30.00 | 25.78 | 25.40 | 29.25 |
| Total surfactant | 30.00 | 30.00 | 35.11 | 30.97 | 30.94 |
| HFO/DPG | 0.65 | 0.65 | 0.55 | 0.54 | 0.53 |
| Bulk Viscosity (cps) | 775 | 522 | 612 | 442 | 100 |
| Foam Density (g/ml) | 0.15 | 0.15 | 0.16 | 0.17 | 0.16 |
| Stability | stable | stable | stable | stable | stable |
| | Ex. 36 | Ex. 37 | Ex. 38 | Ex. 39 | Ex. 40 |
| Sodium laureth-1-sulfate | 6.83 | 25.73 | 17.59 | 12.28 | 7.19 |
| SLE1S¹ | | | | | |
| Branched sodium trideceth-2- | 21.46 | 3.99 | 5.37 | 17.03 | 19.40 |
| sulfate ST2S² | | | | | |
| Sodium Undecyl Sulfate (C11 | | | | | |
| 70% active)³ | | | | | |
| Cocoamidopropyl betaine⁴ | 2.21 | 2.29 | 5.17 | 3.92 | 1.72 |
| Sodium Lauroamphoacetate⁵ | 3.27 | 0.67 | 3.83 | 1.53 | 5.44 |
| Lauramidopropyl Betaine | | | | | |
| (LAPB 35% active)⁶ | | | | | |
| Dipropylene glycol | 10.55 | 10.90 | 11.11 | 12.38 | 13.19 |
| Guar, Hydroxylpropyl | | | | | |
| Trimonium Chloride, Jaguar | | | | | |
| C-500⁷ | | | | | |
| Guar, Hydroxylpropyl | | | | | |
| Trimonium Chloride, N- | | | | | |
| Hance 3196⁸ | | | | | |
| Silicone quaternium⁹ | | | | | |
| Perfume | 1.73 | 1.81 | 2.02 | 1.98 | 2.23 |
| HFO¹⁰ | 5.2 | 5.2 | 5.2 | 5.2 | 5.2 |
| Preservatives, pH adjusters | Up to 1% | Up to 1% | Up to 1% | Up to 1% | Up to 1% |
| Water | Q.S. | Q.S. | Q.S. | Q.S. | Q.S. |
| Total anionic Surfactant | 28.29 | 29.72 | 22.95 | 29.31 | 26.59 |
| Total surfactant | 33.77 | 32.68 | 31.96 | 34.76 | 33.75 |
| HFO/DPG | 0.49 | 0.48 | 0.47 | 0.42 | 0.40 |
| Bulk Viscosity (cps) | 124 | 597 | 250 | 151 | 84 |
| Foam Density (g/ml) | 0.16 | 0.15 | 0.15 | 0.15 | 0.15 |
| Stability | stable | stable | stable | stable | stable |
| | Ex. 41 | Ex. 42 | Ex. 43 | Ex. 44 | Ex. 45 |
| Sodium laureth-1-sulfate | 22.95 | 4.45 | 15.14 | 28.25 | 17.91 |
| SLE1S¹ | | | | | |
| Branched sodium trideceth-2- | 3.00 | 19.14 | 11.29 | 0.71 | 6.27 |
| sulfate ST2S² | | | | | |
| Sodium Undecyl Sulfate (C11 | | | | | |
| 70% active)³ | | | | | |
| Cocoamidopropyl betaine⁴ | 1.45 | 7.87 | 0.91 | 7.76 | 7.18 |
| Sodium Lauroamphoacetate⁵ | 3.98 | 1.17 | 5.88 | 1.29 | 2.81 |
| Lauramidopropyl Betaine | | | | | |
| (LAPB 35% active)⁶ | | | | | |
| Dipropylene glycol | 13.63 | 13.80 | 14.06 | 15.28 | 15.44 |
| Guar, Hydroxylpropyl | | | | | |
| Trimonium Chloride, Jaguar | | | | | |
| C-500⁷ | | | | | |
| Guar, Hydroxylpropyl | | | | | |
| Trimonium Chloride, N- | | | | | |
| Hance 3196⁸ | | | | | |
| Silicone quaternium⁹ | | | | | |
| Perfume | 1.76 | 1.83 | 1.83 | 2.06 | 1.66 |
| HFO¹⁰ | 5.2 | 5.2 | 5.2 | 5.2 | 5.2 |
| Preservatives, pH adjusters | Up to 1% | Up to 1% | Up to 1% | Up to 1% | Up to 1% |
| Water | Q.S. | Q.S. | Q.S. | Q.S. | Q.S. |
| Total anionic Surfactant | 25.95 | 23.59 | 26.43 | 28.97 | 24.18 |
| Total surfactant | 31.39 | 32.62 | 33.22 | 38.01 | 34.17 |
| HFO/DPG | 0.38 | 0.38 | 0.37 | 0.34 | 0.34 |
| Bulk Viscosity (cps) | 142 | 102 | 102 | 359 | 149 |
| Foam Density (g/ml) | 0.15 | 0.15 | 0.14 | 0.15 | 0.15 |
| Stability | stable | stable | stable | stable | stable |
| | Ex. 46 | Ex. 47 | Ex. 48 | Ex. 49 | Ex. 50 |
| Sodium laureth-1-sulfate | 18.35 | 5.49 | 30.00 | 30.00 | 24.00 |
| SLE1S¹ | | | | | |
| Branched sodium trideceth-2- | 9.80 | 16.05 | 0.00 | 0.00 | 0.00 |
| sulfate ST2S² | | | | | |
| Sodium Undecyl Sulfate (C11 | | | | | |
| 70% active)³ | | | | | |
| Cocoamidopropyl betaine⁴ | 2.85 | 6.08 | 0.00 | 0.00 | 0.00 |
| Sodium Lauroamphoacetate⁵ | 0.77 | 5.86 | 0.00 | 0.00 | 6.00 |
| Lauramidopropyl Betaine | | | | | |
| (LAPB 35% active)⁶ | | | | | |
| Dipropylene glycol | 15.64 | 15.82 | 16.00 | 16.00 | 16.00 |
| Guar, Hydroxylpropyl | | | | | |
| Trimonium Chloride, Jaguar | | | | | |
| C-500⁷ | | | | | |
| Guar, Hydroxylpropyl | | | | | |
| Trimonium Chloride, N- | | | | | |
| Hance 3196⁸ | | | | | |
| Silicone quaternium⁹ | | | | | |
| Perfume | 2.24 | 2.11 | 2.40 | 1.60 | 2.00 |
| HFO¹⁰ | 5.2 | 5.2 | 5.2 | 5.2 | 5.2 |
| Preservatives, pH adjusters | Up to 1% | Up to 1% | Up to 1% | Up to 1% | Up to 1% |
| Water | Q.S. | Q.S. | Q.S. | Q.S. | Q.S. |
| Total anionic Surfactant | 28.15 | 21.54 | 30.00 | 30.00 | 24.00 |
| Total surfactant | 31.77 | 33.48 | 30.00 | 30.00 | 30.00 |
| HFO/DPG | 0.33 | 0.33 | 0.33 | 0.33 | 0.33 |
| Bulk Viscosity (cps) | 102 | 84 | 70 | 60 | 76 |
| Foam Density (g/ml) | 0.15 | 0.15 | 0.15 | 0.15 | 0.15 |
| Stability | stable | stable | stable | stable | stable |
| | Ex. 51 | Ex. 52 | Ex. 53 | Ex. 54 | Ex. 55 |
| Sodium laureth-1-sulfate | 20.00 | 20.00 | 20.00 | 20.00 | 15.00 |
| SLE1S¹ | | | | | |
| Branched sodium trideceth-2- | 0.00 | 0.00 | 0.00 | 0.00 | 15.00 |
| sulfate ST2S² | | | | | |
| Sodium Undecyl Sulfate (C11 | | | | | |
| 70% active)³ | | | | | |
| Cocoamidopropyl betaine⁴ | 10.00 | 4.00 | 10.00 | 4.00 | 0.00 |
| Sodium Lauroamphoacetate⁵ | 0.00 | 6.00 | 0.00 | 6.00 | 0.00 |
| Lauramidopropyl Betaine | | | | | |
| (LAPB 35% active)⁶ | | | | | |
| Dipropylene glycol | 16.00 | 16.00 | 16.00 | 16.00 | 16.00 |
| Guar, Hydroxylpropyl | | | | | |
| Trimonium Chloride, Jaguar | | | | | |
| C-500⁷ | | | | | |
| Guar, Hydroxylpropyl | | | | | |
| Trimonium Chloride, N- | | | | | |
| Hance 3196⁸ | | | | | |
| Silicone quaternium⁹ | | | | | |
| Perfume | 2.40 | 1.60 | 1.60 | 2.40 | 2.40 |
| HFO¹⁰ | 5.2 | 5.2 | 5.2 | 5.2 | 5.2 |
| Preservatives, pH adjusters | Up to 1% | Up to 1% | Up to 1% | Up to 1% | Up to 1% |
| Water | Q.S. | Q.S. | Q.S. | Q.S. | Q.S. |
| Total anionic Surfactant | 20.00 | 20.00 | 20.00 | 20.00 | 30.00 |
| Total surfactant | 30.00 | 30.00 | 30.00 | 30.00 | 30.00 |
| HFO/DPG | 0.33 | 0.33 | 0.33 | 0.33 | 0.33 |
| Bulk Viscosity (cps) | 227 | 103 | 161 | 100 | 67 |
| Foam Density (g/ml) | 0.17 | 0.15 | 0.15 | 0.15 | 0.15 |
| Stability | stable | stable | stable | stable | stable |
| | Ex. 56 | Ex. 57 | Ex. 58 | Ex. 59 | Ex. 60 |
| Sodium laureth-1-sulfate | 14.41 | 0.00 | 0.00 | 0.00 | 0.00 |
| SLE1S¹ | | | | | |
| Branched sodium trideceth-2- | 12.59 | 20.00 | 20.00 | 24.00 | 24.00 |
| sulfate ST2S² | | | | | |
| Sodium Undecyl Sulfate (C11 | | | | | |
| 70% active)³ | | | | | |
| Cocoamidopropyl betaine⁴ | 0.00 | 7.00 | 10.00 | 0.00 | 0.00 |
| Sodium Lauroamphoacetate⁵ | 3.00 | 3.00 | 0.00 | 6.00 | 6.00 |
| Lauramidopropyl Betaine | | | | | |
| (LAPB 35% active)⁶ | | | | | |
| Dipropylene glycol | 16.00 | 16.00 | 16.00 | 16.00 | 16.00 |
| Guar, Hydroxylpropyl | | | | | |
| Trimonium Chloride, Jaguar | | | | | |
| C-500⁷ | | | | | |
| Guar, Hydroxylpropyl | | | | | |
| Trimonium Chloride, N- | | | | | |
| Hance 3196⁸ | | | | | |
| Silicone quaternium⁹ | | | | | |
| Perfume | 1.60 | 2.40 | 1.60 | 2.40 | 1.60 |
| HFO¹⁰ | 5.2 | 5.2 | 5.2 | 5.2 | 5.2 |
| Preservatives, pH adjusters | Up to 1% | Up to 1% | Up to 1% | Up to 1% | Up to 1% |
| Water | Q.S. | Q.S. | Q.S. | Q.S. | Q.S. |
| Total anionic Surfactant | 27.00 | 20.00 | 20.00 | 24.00 | 24.00 |
| Total surfactant | 30.00 | 30.00 | 30.00 | 30.00 | 30.00 |
| HFO/DPG | 0.33 | 0.33 | 0.33 | 0.33 | 0.33 |
| Bulk Viscosity (cps) | 62 | 61 | 75 | 46 | 47 |
| Foam Density (g/ml) | 0.15 | 0.15 | 0.15 | 0.15 | 0.14 |
| Stability | stable | stable | stable | stable | stable |

| | Ex. 61 | Ex. 62 | Ex. 63 | Ex. 64 | Ex. 65 | Ex. 66 |
|---|---|---|---|---|---|---|
| Sodium laureth-1-sulfate | 0.00 | 0.00 | 0.00 | 18 | 18 | |
| SLE1S¹ | | | | | | |
| Branched sodium trideceth-2- | 25.00 | 30.00 | 30.00 | 6 | 8 | |
| sulfate ST2S² | | | | | | |
| Sodium Undecyl Sulfate (C11 | | | | | | |
| 70% active)³ | | | | | | |
| Cocoamidopropyl betaine⁴ | 5.00 | 0.00 | 0.00 | | 2 | |
| Sodium Lauroamphoacetate⁵ | 0.00 | 0.00 | 0.00 | | 2 | |
| Lauramidopropyl Betaine | | | | | | |
| (LAPB 35% active)⁶ | | | | | | |
| Disodium Laureth | | | | | | 10 |
| sulfosuccinate¹² | | | | | | |
| Coco Glucoside¹³ | | | | | | 9.8 |
| Sodium cocoamphoacetate¹⁴ | | | | | | 11.85 |
| Dipropylene glycol | 16.00 | 16.00 | 16.00 | 4 | 4 | 4 |
| Guar, Hydroxylpropyl | | | | | 0.4 | 0.4 |
| Trimonium Chloride, Jaguar | | | | | | |
| C-500⁷ | | | | | | |
| Guar, Hydroxylpropyl | | | | | | |
| Trimonium Chloride, N- | | | | | | |
| Hance 3196⁸ | | | | | | |
| Piroctone Olamine¹¹ | | | | 1.0 | 2.0 | |
| Perfume | 2.00 | 2.40 | 1.60 | 1.50 | 2.40 | 2.40 |
| HFO¹⁰ | 5.2 | 5.2 | 5.2 | 6.7 | 6.7 | 7.0 |
| Preservatives, pH adjusters | Up to 1% | Up to 1% | Up to 1% | Up to 1% | Up to 1% | Up to 1% |
| Water | Q.S. | Q.S. | Q.S. | Q.S. | Q.S. | Q.S. |
| Total anionic Surfactant | 25.00 | 30.00 | 30.00 | 24 | 26.00 | 10.00 |
| Total surfactant | 30.00 | 30.00 | 30.00 | 24 | 30.00 | 30.00 |
| HFO/DPG | 0.33 | 0.33 | 0.33 | 1.67 | 1.67 | 1.75 |
| Bulk Viscosity (cps) | 60 | 42 | 43 | 1960 | 2029 | 2196 |
| Foam Density (g/ml) | 0.15 | 0.15 | 0.15 | 0.11 | 0.11 | 0.10 |
| Stability | stable | stable | stable | stable | stable | stable |

| | Ex. 67 | Ex. 68 | Ex. 69 | Ex. 70 |
|---|---|---|---|---|
| Sodium lauroyl methyl | 12.0 | 11.33 | 10.0 | — |
| isethionate¹⁵ | | | | |
| Sodium cocoyl isethionate¹⁶ | 5.0 | 4.72 | 5.0 | — |
| Sodium lauroyl glycinate¹⁷ | — | 9.45 | — | — |
| Disodium cocoyl glutamate¹⁸ | — | — | 5.0 | — |
| Sodium lauroyl sarcosinate¹⁹ | — | — | — | 20.0 |
| Cocoamidopropyl betaine⁴ | 8.0 | 1.9 | — | 10.0 |
| Lauramidopropyl Betaine | — | — | 5.0 | — |
| (LAPB 35% active)⁶ | | | | |
| Dipropylene glycol | 4.67 | 3.74 | 2.0 | 2.5 |
| Guar, Hydroxylpropyl | 0.50 | — | — | 0.25 |
| Trimonium Chloride, Jaguar | | | | |
| C-500⁷ | | | | |
| Guar, Hydroxylpropyl | — | 0.33 | 0.35 | — |
| Trimonium Chloride, Jaguar | | | | |
| Optima²⁰ | | | | |
| Polyquaternium 6²¹ | — | — | 0.2 | — |
| Versene ™ 220²² | 0.16 | 0.16 | 0.16 | 0.17 |
| Natrlquest E30²³ | 0.23 | 0.23 | 0.23 | 0.23 |
| Sodium benzoate | 0.24 | 0.24 | 0.24 | 0.24 |
| Kathon ™²⁴ | 0.03 | 0.03 | 0.03 | 0.03 |
| Perfume | 0.90 | 0.90 | 2.0 | 0.90 |
| DL-Panthanol 50L²⁵ | 0.05 | 0.05 | 0.05 | 0.05 |
| D/DI Panthenyl ether²⁶ | 0.03 | 0.03 | 0.03 | 0.03 |
| HFO¹⁰ | 5.5 | 5.5 | 5.5 | 5.5 |
| Water | Q.S. | Q.S. | Q.S. | Q.S. |
| Total anionic Surfactant | 17.0 | 25.5 | 20.0 | 20.0 |
| Total surfactant | 25.0 | 27.4 | 25.0 | 30.0 |
| HFO/DPG | 1.18 | 1.47 | 2.75 | 2.2 |
| Bulk viscosity, (cps) | 1273 | 658 | 935 | 520 |
| Stability | Stable | stable | stable | Stable |

### Comparative Examples

| | Comp. | Comp. | Comp. | Comp. | Comp. | Comp. |
|---|---|---|---|---|---|---|
| | Ex. 1 | Ex. 2 | Ex. 3 | Ex. 4 | Ex. 5 | Ex. 6 |
| Sodium laureth-1-sulfate | | | | | 18 | |
| SLE1S¹ | | | | | | |
| Branched sodium trideceth-2- | | | | | 8 | |
| sulfate ST2S² | | | | | | |
| Sodium Undecyl Sulfate (C11 | 24 | 24 | 12 | 12 | | 24 |
| 70% active)³ | | | | | | |
| Cocoamidopropyl betaine⁴ | | | | | 2 | |
| Sodium Lauroamphoacetate⁵ | | | | | 2 | |
| Lauramidopropyl Betaine | 6 | 6 | 3 | 3 | | 6 |
| (LAPB 35% active)⁶ | | | | | | |
| Dipropylene glycol | 4 | 4 | 2 | 2 | 4 | 4 |
| Guar, Hydroxylpropyl | 0.40 | 0.40 | 0.20 | 0.20 | 0.40 | 0.40 |
| Trimonium Chloride, Jaguar | | | | | | |
| C-500⁷ | | | | | | |
| Guar, Hydroxylpropyl | | | | | | |
| Trimonium Chloride, N- | | | | | | |
| Hance 3196⁸ | | | | | | |
| Silicone quaternium⁹ | | | | | | |
| Perfume | 2.40 | 2.40 | 1.20 | 1.20 | 2.40 | 2.40 |
| HFO¹⁰ | 1.2 | 2.1 | 1.7 | 2.8 | 2.2 | 13.2 |
| Preservatives, pH adjusters | Up to 1% | Up to 1% | Up to 1% | Up to 1% | Up to 1% | Up to 1% |
| Water | Q.S. | Q.S. | Q.S. | Q.S. | Q.S. | Q.S. |
| Total anionic Surfactant | 24.00 | 24.00 | 12.00 | 12.00 | 26.00 | 24.00 |
| Total surfactant | 30.00 | 30.00 | 15.00 | 15.00 | 30.00 | 30.00 |
| HFO/DPG | 0.30 | 0.54 | 0.86 | 1.38 | 0.54 | 3.31 |
| Bulk Viscosity (cps) | 556 | 556 | 50 | 50 | 2029 | 556 |
| Foam Density (g/ml) | 0.72 | 0.40 | 0.59 | 0.36 | 0.47 | 0.04 |
| Stability | stable | stable | stable | stable | stable | 2 phase |

| | Comp. | Comp. | Comp. | Comp. | Comp. |
|---|---|---|---|---|---|
| | Ex. 7 | Ex. 8 | Ex. 9 | Ex. 10 | Ex. 11 |
| Sodium laureth-1-sulfate | | | | | 18 |
| SLE1S¹ | | | | | |
| Branched sodium trideceth-2- | | | | | 8 |
| sulfate ST2S² | | | | | |
| Sodium Undecyl Sulfate (C11 | 12 | 12 | 12 | 12 | |
| 70% active)³ | | | | | |
| Cocoamidopropyl betaine⁴ | | | | | 2 |
| Sodium Lauroamphoacetate⁵ | | | | | 2 |
| Lauramidopropyl Betaine | 3 | 3 | 3 | 3 | |
| (LAPB 35% active)⁶ | | | | | |
| Dipropylene glycol | 2 | 2 | 2 | 2 | 4 |
| Guar, Hydroxylpropyl | 0.20 | 0.20 | 0.20 | 0.20 | 0.40 |
| Trimonium Chloride, Jaguar | | | | | |
| C-500⁷ | | | | | |
| Guar, Hydroxylpropyl | | | | | |
| Trimonium Chloride, N- | | | | | |
| Hance 3196⁸ | | | | | |
| Silicone quaternium⁹ | | | | | |
| Perfume | 1.20 | 1.20 | 1.20 | 1.20 | 2.40 |
| HFO¹⁰ | 8.6 | 10.7 | 12.4 | 13.9 | 13.6 |
| Preservatives, pH adjusters | Up to 1% | Up to 1% | Up to 1% | Up to 1% | Up to 1% |
| Water | Q.S. | Q.S. | Q.S. | Q.S. | Q.S. |
| Total anionic Surfactant | 12.00 | 12.00 | 12.00 | 12.00 | 26.00 |
| Total surfactant | 15.00 | 15.00 | 15.00 | 15.00 | 30.00 |
| HFO/DPG | 4.29 | 5.37 | 6.20 | 6.95 | 3.40 |
| Bulk Viscosity (cps) | 50 | 50 | 50 | 50 | 2029 |
| Foam Density (g/ml) | 0.06 | 0.05 | 0.04 | 0.03 | 0.05 |
| Stability | 2 phase | 2 phase | 2 phase | 2 phase | 2 phase |

¹Sodium Laureth (1 molar ethylene oxide) sulfate at 70% active, supplier: Stephan Co
²Sodium Tridecyl Ether Sulfate (2 molar ethylene oxide), Stepan ST2S-65 (Steol-TD 402 65) 65% active, supplier: Stephan Co
³Sodium Undecyl Sulfate (C11, Isachem 123S) at 70% active, supplier: P&G
⁴Cocamidopropyl betaine, trade name: Amphosol HCA-HP, 30.0% active from Stepan
⁵Sodium Lauroamphoacetate, NaLaa (Miranol Ultra L32) at 32% active level, supplier: Solvay
⁶Lauramidopropyl Betaine, LAPB (Mackam DAB), at 35% active level, supplier: Solvay
⁷Jaguar C500, MW of 500,000, CD of 0.7 meg/g, from Solvay
⁸N-Hance 3196 MW of 1,700,000, CD of 0.7 meg/g, from Ashland
⁹Silicone quaternium micro-emulsion, 30% active, Abil ME 45, from Evonik
¹⁰Blowing Agent HFO (Trans 1,3,3,3-tetrafluoroprop-1-ene), Solstice ® HFO-1234ze from Honey Well
¹¹Octopirox (Piroctone Olamine) from Clariant
¹²Disodium Laureth Sulfosuccinate, Texapon SB 3, 40% active, from BASF
¹³Coco Glucoside, Plantaren 818 UP, C8-16 fatty alcohol glucoside, 52% active, from BASF
¹⁴Sodium Cocoamphoacetate (NaCaa), Dehyton MC, 39% active, from BASF
¹⁵Sodium Lauroyl methyl isethionate, tradename: Iselux from Innospec
¹⁶Sodium cocoyl isethionate, tradename: Jordapon CI Prill from BASF
¹⁷Sodium lauroyl glucinate, trade name: Pureact SLG from Innospec
¹⁸Disodium cocly glutamate, trade name: Eversoft UCS-50SG from Sino Lion.
¹⁹Soduim lauroyl sarcosinate from Croda
²⁰Jaguar Optima MW of 500,000, CD of 1.25 meg/g, from Solvay
²¹Polyquaternium 6, PolyDADMAC, MW of 150,000, CD of 6.2 meg/g, tradename: Mirapol ® 100s, 31.5% active, from Solvay
²²Versene ™ 220, Tetrasodium ethylenediaminetetraacetate tetrahydrate from Dow
²³Natrlquest E30, Trisodium Ethylenediamine Disuccinate, from Innospec.
²⁴Kathon ™CG, from Dow
²⁵DL-Panthanol 50L from DSM Nutritional Products
²⁶D/DI Panthenyl ether from DSM Nutritional Products

### Combinations

Paragraph A. A hair care composition comprising:
a) from about 5 weight % to about 45 weight % total surfactant;
b) from about 5 weight % to about 35 weight % anionic surfactant
c) from about 1 to about 20% by weight of a water miscible solvent;
d) greater than about 3 weight % HFO;
wherein the ratio of HFO to water miscible solvent is lower than about 3.
Paragraph B. The hair care composition of Paragraph A having a foam density of from about 0.03 to about 0.30 g/mL.
Paragraph C. The hair care composition of Paragraphs A-B, having viscosity of about 1 to about 3000 cP at 26.5° C.
Paragraph D. The hair care composition of Paragraphs A-C, wherein the composition has a viscosity of about 10 to about 3000 cP at 26.5° C.
Paragraph E. The hair care composition of Paragraphs A-D, wherein the composition has a viscosity of about 50 to about 2000 cP at 26.5° C.
Paragraph F. The hair care composition of Paragraphs A-E, wherein the concentration of the HFO is from about 3 to about 12 weight %.
Paragraph G. The hair care composition of Paragraphs A-F, wherein the composition further comprises from about 0.1 weight % to about 25 weight % one or more co-surfactants selected from the group consisting of amphoteric surfactant, zwitterionic surfactant, non-ionic surfactant and mixtures thereof.
Paragraph H. The hair care composition of Paragraphs A-G, wherein the composition further comprises from about 2 weight % to about 20 weight % one or more co-surfactants selected from the group consisting of amphoteric surfactant, zwitterionic surfactant, non-ionic surfactant and mixtures thereof. Paragraph I. The hair care composition of Paragraphs A-H, wherein the concentration of the water miscible agent is from about 2 to about 20 weight %.
Paragraph J. The hair care composition of Paragraphs A-I, wherein the hair care composition further contains about 0.05 to 5 weight percent of a silicone conditioning agent.
Paragraph K. The hair care composition of Paragraphs A-J, wherein the silicone conditioning agent contains one of more quaternary ammonium salt in its molecular structure.
Paragraph L. The hair care composition of Paragraphs A-K, wherein the silicone conditioning agent is dimethiconol micro-emulsion.
Paragraph M. The hair care composition of Paragraphs A-L, wherein the hair care composition further comprises from about 0.05 to about 2 weight % of the hair care composition of one or more cationic polymers.
Paragraph N. The hair care composition of Paragraphs A-M, wherein the concentration of the cationic polymer is from about 0.05 to about 1 weight % of the composition.
Paragraph O. The hair care composition of Paragraphs A-N, wherein the cationic polymers are selected from the group consisting of guar hydroxylpropyltrimonium chloride, Polyquaternium-6, Polyquaternium-7, Polyquaternium-10, Polyquaternium-39, Polyquaternium-67, and mixtures thereof.
Paragraph P. The hair care composition of Paragraphs A-O, wherein the guar hydroxylpropyltrimonium chloride has a weight average molecular weight of from about 100,000 to about 900,000 g/m.
Paragraph Q. The hair care composition of Paragraphs A-P, wherein the total surfactant level is from about 20% to about 45% by weight.
Paragraph R. The hair care composition of Paragraphs A-Q, wherein the total surfactant level is from about 25% to about 35% by weight.
Paragraph S. The hair care composition of Paragraphs A-R, wherein the anionic surfactant is selected from the group consisting of sodium trideceth sulfate, sodium tridecyl sulfate, sodium C12-13 alkyl sulfate, sodium C12-15 alkyl sulfate, sodium C12-18 alkyl sulfate, sodium C12-13 pareth sulfate, sodium C12-13 pareth-n sulfate, sodium C12-14 pareth-n sulfate, and combinations thereof.
Paragraph T. The hair care composition of Paragraphs A-S, wherein the anionic surfactant is selected from the group consisting of ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, sodium trideceth-1 sulfate, sulfate, sodium trideceth-2 sulfate, sulfate, sodium trideceth-3 sulfate, sodium tridecyl sulfate, sodium methyl lauroyl taurate, sodium methyl cocoyl taurate, sodium lauroyl isethionate, sodium cocoyl isethionate, sodium laurethsulfosuccinate, sodium laurylsulfosuccinate, sodium tridecyl benzene sulfonate, sodium dodecyl benzene sulfonate, and mixtures thereof.
Paragraph U. The hair care composition of Paragraphs A-T, wherein the water miscible solvent is selected from the group consisting of dipropylene glycol, tripropylene glycol, diethylene glycol, ethylene glycol, propylene glycol, glycerin, 1,3-propane diol, 2,2-propanediol, 1,2-butanediol, 1,3-butanediol, 1,4-butanediol, 2,3-butanediol, 2-methyl-2,4-pentanediol, and mixtures thereof.
Paragraph V. The hair care composition of Paragraphs A-U, wherein the hair care composition further comprises about 0.5 to 7 weight percent of a perfume.
Paragraph W. A method of treating the hair, the method comprising:
a. providing a hair care composition of Paragraphs A-V,
b. dispensing the hair care composition from the aerosol foam dispenser as a foam; wherein the foam has a density of from about 0.03 to about 0.30 g/mL when dispensed from the aerosol foam dispenser.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair care composition comprising:
a) from about 5 weight % to about 45 weight % total surfactant;
b) from about 5 weight % to about 35 weight % anionic surfactant
c) from about 1 to about 20% by weight of a water miscible solvent;
d) greater than about 3 weight % HFO;
wherein the ratio of HFO to water miscible solvent is lower than about 3.

2. The hair care composition of claim 1, having a foam density of from about 0.03 to about 0.35 g/mL.

3. The hair care composition of claim 1, having viscosity of about 1 to about 3000 cP at 26.5° C.

4. The hair care composition of claim 1, wherein the concentration of the HFO is from about 3 to about 12 weight %.

5. The hair care composition of claim 1, wherein the composition further comprises from about 0.1 weight % to about 25 weight % one or more co-surfactants selected from the group consisting of amphoteric surfactant, zwitterionic surfactant, non-ionic surfactant and mixtures thereof.

6. The hair care composition of claim 1, wherein the composition further comprises from about 2 weight % to about 20 weight % one or more co-surfactants selected from the group consisting of amphoteric surfactant, zwitterionic surfactant, non-ionic surfactant and mixtures thereof.

7. The hair care composition of claim 1, wherein the hair care composition further contains about 0.05 to 5 weight percent of a silicone conditioning agent.

8. The hair care composition of claim 7, wherein the silicone conditioning agent contains one of more quaternary ammonium salt in its molecular structure.

9. The hair care composition of claim 7, wherein the silicone conditioning agent is dimethiconol micro-emulsion.

10. The hair care composition of claim 1, wherein the composition further comprises from about 0.1 weight % to about 5 weight % of one or more anti-dandruff active.

11. The hair care composition of claim 10, wherein the anti-dandruff active is selected from the group containing piroctone olamine, climbazole, and salicylic acid and mixtures thereof.

12. The hair care composition of claim 1 wherein the hair care composition further comprises from about 0.05 to about 2 weight % of the hair care composition of one or more cationic polymers.

13. The hair care composition of claim 12, wherein the cationic polymers are selected from the group consisting of guar hydroxylpropyltrimonium chloride, Polyquaternium-6, Polyquaternium-7, Polyquaternium-10, Polyquaternium-39, Polyquaternium-67, and mixtures thereof.

14. The hair care composition of claim 13, wherein the guar hydroxylpropyltrimonium chloride has a weight average molecular weight of from about 100,000 to about 2,000,000 g/m and a charge density from about 0.2 to about 2.2 meg/g.

15. The hair care composition of claim 17, wherein the total surfactant level is from about 20% to about 35% by weight.

16. The hair care composition of claim 1, wherein the anionic surfactant is selected from the group consisting of sodium trideceth sulfate, sodium tridecyl sulfate, sodium C12-13 alkyl sulfate, sodium C12-15 alkyl sulfate, sodium C12-18 alkyl sulfate, sodium C12-13 pareth sulfate, sodium C12-13 pareth-n sulfate, sodium C12-14 pareth-n sulfate, and combinations thereof.

17. The hair care composition of claim 1, wherein the anionic surfactant is selected from the group consisting of ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, sodium trideceth-1 sulfate, sulfate, sodium trideceth-2 sulfate, sulfate, sodium trideceth-3 sulfate, sodium tridecyl sulfate, lauryl sarcosine, cocoyl sarcosine, sodium lauryl sarcosinate, sodium lauroyl sarcosinate, acyl sarcosinate, acyl taurate, sodium methyl lauroyl taurate, sodium methyl cocoyl taurate, acyl isethionate, acyl methyl isethionate, sodium lauroyl isethionate, sodium lauroyl methyl isethionate, sodium cocoyl isethionate, sulfosuccinate, sodium laureth sulfosuccinate, sodium lauryl sulfosuccinate, disodium laureth sulfosuccinate, disodium lauryl sulfosuccinate, alpha olefin sulfonate, alkyl benzene sulfonate, sodium tridecyl benzene sulfonate, sodium dodecyl benzene sulfonate, acyl glutamate, sodium cocoyl glutamate, disodium cocoyl glutamate, sodium lauroyl glutamate, disodium lauroyl glutamate, acyl glycinate, sodium lauroyl glycinate, sodium cocoyl glycinate, acyl alaninate, sodium lauroyl alaninate, alkyl ether carboxylate, glucose carboxylate, sodium cocoyl glucose carboxylate, alkylamphoacetate, sodium lauryl sodium cocoyl amphoacetate, sodium lauroyl amphoacetate, and mixtures thereof.

18. The hair care composition of claim 1, wherein the water miscible solvent is selected from the group consisting of dipropylene glycol, tripropylene glycol, diethylene glycol, ethylene glycol, propylene glycol, glycerin, 1,3-propane diol, 2,2-propanediol, 1,2-butanediol, 1,3-butanediol, 1,4-butanediol, 2,3-butanediol, 2-methyl-2,4-pentanediol, and mixtures thereof.

19. The hair care composition of claim 1, wherein the hair care composition further comprises about 0.5 to about 7 weight percent of a perfume.

20. A method of treating the hair, the method comprising:
a. providing a concentrated hair care composition in an aerosol foam dispenser, wherein the concentrated hair care composition comprises:
1) from about 5 weight % to about 45 weight % total surfactant;
2) from about 5 weight % to about 35 weight % anionic surfactant
3) from about 1 to about 20% by weight of a water miscible solvent;
4) from about 3 to about 12 weight % HFO;
wherein the ratio of HFO to water miscible solvent is lower than about 3;
wherein the concentrated hair care composition has viscosity of about 1 to about 3000 cP at 26.5° C.;
b. dispensing the concentrated hair care composition from the aerosol foam dispenser as a foam; wherein the foam has a density of from about 0.03 to about 0.30 g/mL when dispensed from the aerosol foam dispenser.
